(19) United States
(12) Patent Application Publication
(10) Pub. No.: US 2005/0226947 A1
(43) Pub. Date: Oct. 13, 2005

(54) Agents for sequestering serum aging factors and uses therefore

(76) Inventor: Dale Kern, Hyde Park, UT (US)

Correspondence Address: Michael F. Krieger;Kirton & McConkie, P.O. Box 45120, Salt Lake City, UT, 84145, US

(21) Appl. No.: 11/049,585

(22) Filed: Feb. 2, 2005

Related U.S. Application Data
(60) Provisional application No. 60/541,615, filed on Feb. 4, 2004.

(51) Int. Cl. A61K 35/78

(52) U.S. Cl. 424/753

(57) ABSTRACT

Methods for the prevention or treatment of disorders and complications of disorders resulting from cell damage caused by an aging-related isoform of NADH oxidase (arNOX) are described. The agent for such inhibition comprises processed various Narcissus tazzeta extracts, preferably IBR-DORMIN®, both alone and in combination with other inhibition agents, including ubiquinones like coenzyme Q. These agents bind arNOX and inhibit the ability of arNOX to generate reactive oxygen species, thereby decreasing the ability of arNOX to generate reactive oxygen species. Such agents, and their methods of administration, as extremely effective as part of anti-aging treatments.

### RELATED APPLICATIONS

This application claims priority to U.S. Patent Application Ser. No. 60/541,615, filed Feb. 4, 2004, entitled “Agents for Inhibiting Serum Aging Factors and Uses Therefor.”

### BACKGROUND

1. Field of the Invention

The invention relates to agents for sequestering serum aging factors, and methods for using the same. More particularly, the invention relates to agents and methods for using the same, to prevent or treat disorders and complications of disorders resulting from cell damage caused by an aging-related isoform of NADH oxidase (arNOX).

2. Background and Related Art

The plasma membrane NADH oxidase (NOX) is a unique cell surface protein with hydroquinone (NADH) oxidase and protein disulfide-thiol interchange activities that normally responds to hormone and growth factors. NOX (or CLOX) are a family of growth related proteins that are associated with aging cells. A hormone-insensitive and drug-responsive form of the NOX designated tNOX has been described that is specific for cancer cells. For example, see U.S. Pat. No. 5,605,810, which is incorporated herein by reference.

The aging-related isoform of NADH oxidase (arNOX) is a member of this family of proteins. The circulating form of arNOX increases markedly in human sera and in lymphocytes of individuals, especially after the age of 65. The arNOX is uniquely characterized by an ability to generate superoxides, which may contribute significantly to aging-related changes including atherogenesis and other action-at-a-distance aging phenomena. Activity of arNOX in aging cells and in sera has been described previously. See, for example, PCT Pub. App. No. WO 00/57871, which is incorporated by reference in its entirety herein.

This model is consistent with the Mitrochondrial Theory of Aging, which holds that during aging, increased reactive oxygen species in mitochondria cause mutations in the mitochondrial DNA and damage mitochondrial components, resulting in senescence. The mitochondrial theory of aging proposes that accumulation of spontaneous somatic mutations of mitochondrial DNA (mtDNA) leads to errors of mtDNAencoded polypeptide chains. (Manczak M et al., J. Neurochem. 2005 February; 92(3):494-504). These errors, occurring in mtDNAencoded polypeptide chains, are stochastic and randomly transmitted during mitochondrial division and cell division. The consequence of these alterations is defective oxidative phosphorylation. Respiratory chain defects may become associated with increased oxidative stress amplifying the original damage (Ozawa, 1995, Biochim. Biophys. Acta 1271:177-189; and Lenaz, 1998, Biochim. Biophys. Acta 1366:53-67). In this view, therefore, mutated mitochondrial DNA, despite being present only in very small quantities in the body, may be the major generator of oxidative stress.

Where accumulation of somatic mutations of mitochondrial DNA (mtDNA) leads to defective oxidative phosphorylation a plasma membrane oxido-reductase (PMOR) system has been suggested to augment survival of mitochondrially deficient cells through regeneration of oxidized pyridine nucleotide. (de Grey, 1997, BioEssays 19:161-166; de Grey, 1998, Anti-Aging Med. 1:53-66; Yoneda et al, 1995, Biochem. Biophys. Res. Comm. 209:723-729; Schon et al., 1996, Cellular Aging and Cell Death, Wiley and Sons, New York, pp. 19-34; Ozawa, 1997, Physiol. Rev. 77:425-464; and Lenaz, 1998, BioFactors 8:195-204). A model to link accumulation of lesions in mtDNA to extracellular responses, such as the oxidation of lipids in low density lipoprotein (LDLs) and the attendant arterial changes, was first proposed with rho° cells (Larm et al., 1994, Biol. Chem. 269:30097-30100; Lawen et al., 1994, Mol. Aspects. Med. 15:s13-s27; de Grey, 1997, BioEssays 19:161-166; and de Grey, 1998, Anti-Aging Med. 1:53-66). Similar studies have been conducted with transformed human cells in culture. (Vaillant et al., 1996, Bioenerg. Biomemb. 28:531-540).

Under conditions where plasma membrane oxidoreductase (PMOR) is over-expressed electrons are transferred from NADH to external acceptors by a defined electron transport chain, resulting in the generation of reactive oxygen species (ROS) at the cell surface. Such cell surface-generated ROS may then propagate an aging cascade originating in mitochondria to both adjacent cells as well as to circulating blood components such as low density lipoproteins. See PCT Pub. App. No. WO 00/57871.

Consequently, there is a need to find agents that reduce the ability of arNOX to generate reactive oxygen species (ROS) for the purposes of reducing or treating the resultant physiological conditions, such as oxidation of lipids in low density lipoprotein (LDLs) and attendant arterial changes. The arNOX activity of aging cells has been shown to be inhibited by co-enzyme Q (ubiquinone). See PCT Published Application Numbers WO 00/57871, WO 01/72318, and WO 01/72319, the disclosures of which are incorporated herein by reference. However, the use of co-enzyme Q is not completely satisfactory for several reasons: it is costly, it oxidizes easily losing its efficacy, and preparations containing coenzyme Q must be specially packaged to prevent loss of function. Thus, while some agents and methods currently exist, which may inhibit arNOX activity, challenges still exist. Accordingly, it would be an improvement in the art to augment or even replace previously disclosed agents and techniques with the agents and techniques disclosed in this invention.

The skin in particular is vulnerable to damage by reactive oxygen species. The skin is made of several layers, or two major layers. The stratum corneum, or epidermis, is the top layer and forms a protective covering for the skin and controls the flow of water and substances in and out of the skin. The dermis is the lower level of the skin and provides the strength, elasticity and the thickness to the skin. The main cell type of the dermis is fibroblasts, which is responsible for synthesis and secretion of all the dermal matrix components such as collagen, elastin and glycosaminoglycans. Collagen provides the strength, elastin the elasticity, and glycosaminoglycans the moistness and plumpness of the skin.

In addition to being damaged by reactive oxygen species the skin is subject to various damaging stressors. The skin may be damaged abused by soaps, emulsifier-based cosmetics, hot water, organic solvents, dermatological disorders, environmental abuse (wind, air conditioning, central heating) or through the normal aging process (chronoaging), which may be accelerated by exposure of skin various external stressors (e.g. photoaging).

“Anti-aging” cosmetic and medical products, which treat or delay the visible signs of actual aging and weathered skin such as wrinkles, lines, sagging, hyperpigmentation and age spots are desirable. Accordingly, there is a demand for effective natural skin treatments and preventative compositions and methods for using the same.

### SUMMARY OF THE INVENTION

The invention relates to agents for sequestering serum aging factors, and methods for using the same. More particularly, the invention relates to agents and methods for using the same, to prevent or treat disorders and complications of disorders resulting from cell damage caused by an aging-related isoform of NADH oxidase (arNOX). In a preferred embodiment the agents of the invention comprise at least one processed Narcissus tazzeta product.

The invention described herein encompasses pharmaceutical compositions, pharmaceutical kits and methods for the prevention or treatment of disorders and complications of disorders resulting from cell damage caused by an aging-related isoform of NADH oxidase (arNOX). The agent for such inhibition in some embodiments of the invention comprise ingredients extracted from various plant species. One embodiment comprises the use of a processed Narcissus tazzeta product. A preferred embodiment of the processed Narcissus tazzeta product is IBR-DORMIN®, which comprises Narcissus tazzeta extract. Another embodiment comprises the use of the processed Narcissus tazzeta product, both alone and in combination with other inhibition agents, including ubiquinones like coenzyme Q, extracts of Shisandra Chinensis, extracts of Lonicera Japonica, and or extract of Fagopyrum Cymosum. Extracts from each of the foregoing may be utilized individually or in combination with other active and inactive ingredients.

The agents of this invention may bind arNOX and inhibit, or otherwise decrease, the ability of arNOX to generate reactive oxygen species. The inhibition of arNOX results in a decrease in the generation of reactive oxygen species by arNOX. A decrease in reactive oxygen species results in a decrease of oxidative damage resulting from said reactive oxygen species. Such agents, and their methods of administration, are an effective part of anti-aging treatments. Thus, one embodiment of the invention described herein encompasses methods of preventing or treating disorders caused by oxidative damage by an aging-specific isoform of NADH oxidase (arNOX).

The invention described herein further encompasses methods for detecting cell-membrane associated arNOX and soluble arNOX in sera. Further, the invention encompasses methods of assaying, screening, and identifying agents that inhibit arNOX, as well as methods using agents comprising processed Narcissus tazzeta products, preferably IBR-DORMIN®, in combination with ubiquinone to inhibit the ability of arNOX to generate reactive oxygen species. These agents may be formulated into pharmaceutical compositions in the prevention and treatment of disorders caused by oxidative damage. The invention described herein further encompasses properties of agents comprising at least one processed Narcissus tazzeta extract. The invention discloses the isolation and characterization of arNOX using agents comprising at least one processed Narcissus tazzeta extract. Additional information about agents comprising at least one processed Narcissus tazzeta extract, including IBR-DORMIN® can be found in U.S. Pat. Nos. 6,635,287 and 6,347,254, the disclosure of which is also incorporated herein by reference.

The pharmaceutical compositions of this invention may comprise varying modes of administration. The modes of administration of compounds comprise capsules, tablets, soft gels, solutions, suppositories, injections, aerosols, or a kit.

The present invention provides compositions comprising active agent(s), which prevent and/or ameliorates skin damage and associated conditions. Further, the invention encompasses methods for utilizing said compositions.

A preferred embodiment of the invention provides active agents from processed plants for the treatment of skin. The active agents prevent and/or ameliorate skin damage and associated conditions. In one embodiment of the invention the processed plant products sequester arNOX activity. In another embodiment of the invention, the processed plant products inhibit radical oxygen species. In another embodiment agents and methods of the invention prevent and/or improve the health of the skin. For example, the agents may improve skin tone, and helps diminish the appearance of fine lines and visible signs of aging. In another embodiment of the invention, the agents positively affects the body's natural production of collagen and elastin. In another embodiment, the agents of the invention minimize the effects of environmental agitators such as pollution, sun, free radicals and stress.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings.

FIG. 1 illustrates periodic variation in the rate of ferricytochrome c reduction. Ferricytochrome c reduction over 90 min showed four sets of maxima (arrows indicate a 24.7 min period) for sera of a 100 y female. The activity with the period length of 24.7 min is much reduced or absent from sera of young individuals. Each maximum was resolved into a doublet pattern indicated by double and single arrows. The doublet pattern was reproduced with three additional serum samples, an 80 y male, an 83 y male and a 98 y female.

FIG. 2 illustrates rate of ferricytochrome c reduction by buffy coats and sera of old and younger individuals. Buffy coat fractions (A-D) and sera (E-H) pooled from 20-40 y (A,B,E,F) and 70-100 y (C,D,G,J,H) individuals were compared. Rates were monitored continuously at intervals of 1.5 min using a SLM Aminco. DW-2000 spectrophotometer in the dual wavelength mode of operation from the increase in absorbance at 550 nm with 540 nm as reference. Maxima separated by ca 25 min are indicated by the single arrows (C,D,G,H). After 45 min of measurement, superoxide dismutase (SOD, 60 μl/60 units) or coenzyme Q in ethanol (30 μl/450 μg) were added at the curved arrows and the measurements were continued for a total of 120 min. The oscillating activity was unique to buffy coats and sera of the aged individuals and was reduced to basal levels by the addition of either superoxide dismutase (SOD) or coenzyme Q.

FIG. 3 illustrates superoxide dismutase (SOD) inhibition of age-related ferricytochrome c reduction. SOD was added to the reaction mixture at the mid-point of the assay. The rates were determined before (solid symbols, solid lines) and after (open symbols, dashed lines) the addition of SOD. Sera were from old (80 to 100 y, circles) or young (20 to 40 y, triangles) subjects. Results are means of 5 to 10 samples±standard deviations. The lack of complete inhibition is explained by the observation that the oscillating age-related oxidase accounts for only about 30% of the total apparent activity even with sera of these very old individuals. The oscillating activity was completely inhibited by SOD (FIG. 2G).

FIG. 4 illustrates coenzyme Q inhibition of aging-related ferricytochrome c reduction. As in FIG. 2 except that the indicated amounts of coenzyme Q were added instead of SOD. Rates were determined before solid symbols, solid lines and after open symbols dashed lines coenzyme Q addition. Sera were from old (80 to 100 y, circles) or young (20 to 40 y, triangles) subjects. The oscillating activity is largely blocked by coenzyme Q addition (FIG. 2H).

FIG. 5 illustrates rates of NADH-cytochrome c reductase activity of pig liver microsomes. When determined for 1 min every 1.5 min over a total of 90 min, the mean rate was 1.2±0.6 μmoles/min/mg protein without any indications of repeating oscillatory patterns.

FIG. 6 illustrates a Western blot of aging-related NOX protein from sera. This Western blot comparing proteinase K digested pooled sera from young individuals (Lane 1,≦35 y females; Lane 2,≦25 y males; Lane 6, 36-45 y females; Lane 7, 36-45 y male) and aged individuals (Lane 3,≧90 y females; Lane 4, 75-85 y males; Lane 5, 75-85 y females). A protein band at ˜22 kD Lanes 3-5. arrow) was elevated in sera of aged individuals. Detection was by polyclonal peptide antisera generated against the C-terminal adenine nucleotide binding region (H-KQEMTGVAGASLEKRWK-OH) of human tNOX.

FIG. 7 illustrates a correlation between band intensity and superoxide formation from sera of both young and old individuals. The correlation was between the intensity of the immunoreactive band at ˜22 kD and the rate of aging-related ferricytochrome c reduction of the same sample prior to electrophoresis. The results combine information from three different blots.

FIG. 8 illustrates the correlation was between the intensity of the immunoreactive band on the Western blots at ˜22 kD and subject age. Data from three independent Western blots from serum samples as illustrated in FIG. 6 were combined.

FIG. 9 illustrates an aging-related ferricytochrome c reduction averaged at 1.5 min intervals over 40 min of the gel region corresponding to ˜22 kD from sera of an aged patient (80 y male). Activity was restored to the material eluted from the gel slice by first incubating with 100 μM GSH in the presence of 150 μM NADH, pH 7.0. to allow for refolding. Hydrogen peroxide was then added to reform disulfide bonds and to initiate the reaction. Comparable regions of the entire gel were assayed but this fraction alone exhibited an oscillating doublet pattern of ferricytochrome c reduction indicated by double and single arrows with a period length of about 25 min.

FIG. 10 illustrates the response of periodic superoxide generation by arNOX of aged transfusion buffy coats to inhibition by IBR-DORMIN® (upper figure) and lack of inhibition by the product Pilinhib (lower figure). The solid arrows show activity maxima with a period length of ca. 25 min. the preparation with Pilinhib showed two sets of maxims neither of which was inhibited. The reaction s were for 45 min without inhibitor. Inhibitor was added at the large open arrows and the reaction continued for another 45 min in the presence of inhibitor.

FIG. 11 illustrates 2-pyridyidithio substrates generating two moles of pyridinethionine per mole of substrate will provide a direct measure of protein disulfide-thiol interchange activity.

FIG. 12 illustrates the total scoring parameter for each patient at each follow-up visit related to the application of vehicle cream applied to the right elbow of each patient.

FIG. 13 illustrates the total scoring parameter for each patient at each follow up visit related to the application of cream comprised of a processed Narcissus tazzeta extract to the left elbow of each patient.

FIG. 14 illustrates the average percent reduction of scoring parameters for each elbow for each follow up visit.

FIGS. 15a-c depict graphically statistical data related to sensory analysis of several concentrations of cosmetic cream, which comprise a processed Narcissus tazzeta extract. In particular 15a depicts perceived resistance against external aggressions, 15b depicts skin sensitivity and 15c depicts skin protection when a placebo, 1% processed Narcissus tazzeta extract cosmetic cream and 3% processed Narcissus tazzeta extract cosmetic cream were applied to test subjects.

FIGS. 16a-c illustrate depict graphically statistical data related to sensory analysis of several concentrations of cosmetic cream, comprising a processed Narcissus tazzeta extract. In particular 16a depicts skin irritability, 16b depicts skin fatigue and 16c depicts skin tautness when a placebo, 1% processed Narcissus tazzeta extract cosmetic cream and 3% processed Narcissus tazzeta extract cosmetic cream were applied to test subjects.

FIGS. 17a-c illustrate depict graphically statistical data related to sensory analysis of several concentrations of cosmetic cream, which comprise a processed Narcissus tazzeta extract. In particular 17a depicts skin comfort, 17b depicts the appearance of little lines on the skin and 17c depicts skin suppleness when a placebo, 1% processed Narcissus tazzeta extract cosmetic cream and 3% processed Narcissus tazzeta extract cosmetic cream were applied to test subjects.

FIG. 18 illustrates the percent evolution of qualitative sensory analysis for several categories of after applying a placebo, 1% processed Narcissus tazzeta extract cosmetic cream and 3% processed Narcissus tazzeta extract cosmetic cream four weeks related to a group of patients.

### DETAILED DESCRIPTION OF THE INVENTION

The invention relates to agents for sequestering serum aging factors, and methods for using the same. More particularly, the invention relates to agents and methods for using the same, to prevent or treat disorders and complications of disorders resulting from cell damage caused by an aging-related isoform of NADH oxidase (arNOX). In a preferred embodiment the agents of the invention comprise at least one processed Narcissus tazzeta product. One embodiment of the invention comprises agents that bind arNOX and inhibit the ability of arNOX to generate reactive oxygen species as well as methods of using these agents to inhibit the ability of arNOX to generate reactive oxygen species.

The invention provides pharmaceutical compositions, methods of use, and pharmaceutical kits for the treatment of disorders resulting from oxidative changes in cells that result in aging by targeting an aging-related isoform of NADH oxidase (arNOX), shed into the sera by aging cells. The compositions may contain agents extracted from plants. For example the compositions of the invention may comprise at least one processed Narcissus tazzeta product, whether alone or with other inhibition agents and inhibit the activity of an aging-related isoform of NADH oxidase shed into the sera by aging cells, wherein the other inhibition agents may comprise ubiquinones, extracts of Shisandra Chinensis, or Lonicera Japonica, or extracts of Fagopyrum Cymosum, In a preferred embodiment the processed Narcissus tazzeta extract is IBR-DORMIN®.

As used herein, the term “disorder” refers to any condition of a living animal or plant body or of one of its parts that impairs normal functioning comprising any ailment, disease, illness, clinical condition, pathological condition, weakened condition, unsound condition, and any abnormal or undesirable physical condition.

As used herein, the term “reactive oxygen species” refers to oxygen derivatives from oxygen metabolism or the transfer of free electrons, resulting in the formation of free radicals (e.g., superoxides or hydroxyl radicals).

As used herein, the term “antioxidant” refers to compounds that neutralize the activity of reactive oxygen species or inhibit the cellular damage done by said reactive species.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment corresponding to the formulations described in this application, which exhibit antioxidant activity, and is relatively non-toxic to the subject.

The term “therapeutic agent” refers to any molecule, compound, or treatment, preferably an antioxidant, which assists in the prevention or treatment of the disorders, or complications of disorders caused by reactive oxygen species.

The term “agent that sequesters arNOX” refers to any molecule, compound, or treatment that interacts with arNOX, thus decreasing the reaction of arNOX with other substrates and inhibits the ability of arNOX to generate reactive oxygen species.

The antioxidants, cellular components, and target proteins defined herein are abbreviated as follows:

| | |
|---|---|
| mitochondrial DNA | mtDNA |
| nicotinamide adenine dinucleotide | NADH |
| cell surface hydroquinone (NADH) oxidase with | NOX |
| protein disulfide-thiol isomerase activity | |
| NOX specific to non-cancer cells | CNOX |
| NOX specific to aged cells | AR-NOX |
| NOX specific to cancer cells | tNOX |
| low density lipoproteins | LDLs |
| plasma membrane oxido-reductase chain | PMOR |
| ubiquinone or coenzyme Q | CoQ |
| coenzyme Q₁₀ | CoQ₁₀ |
| reactive oxygen species | ROS |

The following disclosure of the present invention is grouped into subheadings, The utilization of the subheadings is for convenience of the reader only and is not to be construed as limiting in any sense.

1. Plasma Membrane Hydroquinone (NADH) Oxidase (NOX)

The plasma membrane NADH oxidase (NOX) is a unique cell surface protein with hydroquinone (NADH) oxidase and protein disulfide-thiol interchange activities that normally responds to hormone- and growth factors. A hormone insensitive and drug-responsive form of the activity designated tNOX also has been described, which is specific for cancer cells. Evidence exists that NOX proteins, under certain conditions, are capable of the production of ROS. For example, ultraviolet light as a source of oxidative stress in cultured cells is used to initiate superoxide generation (Morré et al., 1999, Biofactors 9:179-187) (See U.S. Pat. No. 5,605,810, which is incorporated by reference in its entirety).

2. Plasma Membrane Levels of Coenzyme Q

Plasma membrane ubiquinone or coenzyme Q (CoQ) plays a major role in the PMOR system. Ubiquinone or coenzyme Q (CoQ) occurs ubiquitously among tissues. The ubiquinone content of plasma membrane is two to five times that of microsomes and only half that of mitochondria. Ubiquinone has long been considered to have both pro- and antioxidant roles over and above its more conventional role in mediating electron transport between NADH and succinic dehydrogenase and the cytochrome system of mitochondria (Emster and Daliner, 1995, Biochim. Biophys. Acta 127:195-204; and Crane and Barr, 1985, Coenzyme Q, John Wiley & Sons, Chichester 1-37).

CoQ is normally a product of cellular biosynthesis and provides a potentially important source of one-electron pro-oxidant oxygen reduction (Anderson et al., 1994, Biochim. Biophys. Acta 1214:79-87; Appelkvist et al., 1994, Molec. Aspects Med. 15S:37-46). In its reduced hydroquinone form (ubiquinol), it is a powerful antioxidant acting directly upon either superoxide or indirectly on lipid radicals alone or together with vitamin E(α-tocopherol) (Crane and Barr, 1985, Coenzyme Q, John Wiley & Sons, Chichester, pp. 1-37; Beyer and Emster, 1990, Highlights of Ubiquinone Research, Taylor & Francis, London, pp. 191-213; Beyer, 1994, J. Bioenerg. Biomemb. 26:349-358; Kagan et al., 1990, Biochem. Biophys. Res. Comm. 169:851-857; and Ernster et al., 1992, BioFactors 3:241-248).

The antioxidant action of ubiquinol normally yields the ubisemiquinone radical. The latter is converted back to ubiquinol by re-reduction through the electron transfer chain in mitochondria, or by various quinone reductases in various cellular compartments including the plasma membrane (Takahashi et al., 1995, Biochem. J. 309:883-890; Takahashi et al., 1996, J. Biochem. (Tokyo) 119:256-263; Beyer et al., 1996, Proc. Natl. Acad. Sci. U.S.A. 93:2528-2532; Beyer et al., 1997, Molec. Aspects Med. 18:slS-s23; Navarro et al., 1995, Biochem. Biophys. Res. Comm. 212:138-143; Villalba et al., 1995, Molec. Aspects Med. 18:s7-s13; and Arroyo et al., 1998, Protoplasma 205:107-113). Thus, ubiquinone may transform from a beneficial one-electron carrier to a superoxide generator if the ubisemiquinone anion becomes protonated (Nohl et al., 1996, Free Rad. Biol. Med. 20:207-15 213).

Exogenous CoQ addition may prevent ROS production and concomitantly protect cells from oxidative damage. For example, exogenous CoQ affects NOX-mediated ROS production. (Valls et al., 1994, Biochem. Mol. Biol. Tnt. 33:633-642; Beyer et al., 1996, Proc. Natl. Acad. Sci. U.S.A. 93:2528-2532; and PCT Pub. App. No. WO 00/5787). The antioxidant effect at the plasma membrane may ameliorate LDL oxidation by scavenging ROS by PMOR produced at the cell surface (Thomas et al., 1997, Molec. Aspects Med. 8:s85-s 103).

Some studies have shown that overall CoQ levels decrease with age (Beyer et al., 1985, Mech. Aging Dev. 32:267-281; Kalen et al., 1990, Lipids 25:93-99; and Genova et al., 1995, Biochem. J. 311:105-109). However, this is not true for all tissues and especially for the brain, where high CoQ levels are maintained throughout aging (Soderberg et al., 1990, J. Neurochem. 54:415-423 and Battino et al., 1995, Mech. Aging Dev. 78:173-187). Thus, the invention also encompasses particular therapeutic levels of coenzyme Q for inhibiting or reducing the effects caused by overactive or aberrant cell surface PMOR system and for sequestering NOX isoforms.

3. Isolation and Characterization of arNOX

The invention encompasses research related to arNOX, an aging isoform of the cell surface NADH oxidase, which is capable of oxidizing reduced quinones. The NOX protein is anchored in the outer leaflet of the plasma membrane (Morre, 1995, Biochem. Biophys. Acta. 1240:201-208; and DeHahn et al., 1997, Biochem. Biophys. Acta. 1328:99-108). NOX activity was shown to be shed in soluble form from the cell surface (Morre et al., 1996, Biochim. Biophys. Acta 1280:197-206). The presence of the shed form in the circulation provides an opportunity to use patient sera as a source of the NOX protein for isolation and characterization studies. A serum form of the CNOX activity specific to sera from elderly subjects (arNOX) has been identified. (PCT Pub. App. No. WO 00/57871).

The invention is based on the identification of arNOX, which is a constitutive cell surface NADH oxidase protein (CNOX) capable of oxidizing reduced quinones. The NOX proteins have been postulated to link the accumulation of lesions in mitochondrial DNA to cell surface accumulations of reactive oxygen species as one consequence of its role as a terminal oxidase in a plasma membrane electron transport chain (Morre, D. M. et al., 2000, J. Expl Biol 203:1513-1521). Cells with functionally deficient mitochondria become characterized by an anaerobic metabolism. NADH accumulated from the glycolytic production of ATP and an elevated plasma membrane electron transport activity become necessary to maintain the NAD⁺/NADH homeostasis essential for survival. Previous findings demonstrate that the hyperactivity of the plasma membrane electron transport system results in an NADH oxidase activity capable of cell surface generation of reactive oxygen species (Morre, D. J. et al., 1999 BioFactors 9:179-187). This would serve to propagate the aging cascade both to adjacent cells and to oxidize circulating lipoproteins.

ArNOX has a superoxide-generating and aging-related enzymatic activity, which is substantially reduced by addition of coenzyme Q and processed Narcissus tazzeta products. A feature of the aging isoform of the NOX protein is that the generation of superoxide by this protein associated with aging is inhibited both by processed Narcissus tazzeta products and by coenzyme Q. These findings provide a rational basis for the anti-aging activity of processed Narcissus tazzeta products with skin and by circulating coenzyme Q in the prevention of atherosclerosis, and other oxidative changes in cell membranes and circulating lipoproteins. Thus, one embodiment of the invention encompasses the findings that arNOX provides a molecular target for processed Narcissus tazzeta products and ubiquinones (coenzyme Q) to offer protection to maintain skin vitality as well as ablate cardiovascular changes associated with cellular aging. Another embodiment of the invention prevents programmed cellular death, apoptosis, by utilizing agents, which sequester, neutralize, bind, or otherwise block or eliminate, the arNOX protein and inhibit its ability to generate reactive oxygen species.

Generally, the characteristics of aged cells includes those that express and/or shed arNOX, and include, but are not limited to, those exhibiting one or more of the following characteristics: an age-related PMOR system, the ability to generate reactive oxygen species, and have functionally defective mitochondria. One embodiment of the invention is the utilization of agents to reduce the negative effects of aging cells.

Another embodiment of the invention is directed to utilizing agents, which switch the NOX protein from oxygen reduction to protein disulfide reduction. For example drugs or supplements may be utilized as agents. The advantage of such an approach has already been observed with plant cells in response to auxins (Chueh et al., 1997, Biol. Chem. 272:11221-1227).

NOX-specific polyclonal antibody to the arNOX protein from lymphocytes have been produced. Once the amino acid sequence of arNOX is deduced from the corresponding cDNA sequence, the amino acid sequence may be used to strategically generate peptide sera with therapeutic potential as probes specific to arNOX to investigate and ameliorate NOX responses to aging. Using methods, which are well known to those skilled in the art, recombinant cDNA libraries may be constructed using RNA prepared from cells known to express arNOX. See, for example, the techniques described in Sambrook et al., 1989, Molecular Cloning: A Laboratory Manual, Cold Spring Harbor Laboratory, N.Y.; and Current Protocols in Molecular Biology, Green Publishing Associates and Wiley Intersciences, N.Y. Alternatively, a human cDNA library may be obtained from a commercial source, e.g., Stratagene. The recombinant cDNA libraries may be screened using a number of different techniques, which are well known to those skilled in the art.

In yet another embodiment of the invention, a cDNA library may be engineered into a mammalian expression vector and screened by transfection into the appropriate mammalian cell line followed by assaying for arNOX activity in the tissue culture supernatant.

In yet another embodiment of the invention, a method for cloning arNOX by means of polymerase chain reaction may be used to clone a cDNA coding for arNOX. Such a method may be utilized using RNA prepared from lymphocytes of aged individuals.

Alternatively, arNOX may be cloned by polymerase chain reaction (PCR) amplification of a human cDNA library obtained from a commercial source (e.g., Stratagene). In addition, gene expression assays using gene expression arrays or microarrays are now practicable for identifying changes in gene expression patterns between different cells or tissue types (see, e.g., Schena et al., 1995, Science 270:467-470; Lockhart et al., 1996, Nature Biotechnology 14:1674-1680; and Blanchard et al., 1996, Nature Biotechnology 14:1649). Thus, in another alternative embodiment of the invention, such gene expression arrays or microarrays may be used to compare mRNA expression patterns in cells that exhibit arNOX activity (e.g., as determined by one of the assays of the present invention) to mRNA expression patterns in cells that do not exhibit arNOX activity and thus, do not express arNOX.

4. Methods of Detecting arNOX

The invention encompasses methods for detecting cell-membrane associated arNOX and soluble arNOX in sera. See, e.g., PCT Pub. App. No. WO 00/57871, which is incorporated by reference in its entirety. The invention further contemplates using arNOX as a diagnostic tool when oxidative damage to cells and/or tissue is suspected. As such, arNOX in tissue, cells, or circulation may be detected. Embodiments include: detection by employing antibodies specific to arNOX, which may be conjugated to a wide variety of labels, wherein the label provides a detectable signal. For example radioisotopes, enzymes, fluorescence and the like may be utilized as labels. Examples of detection techniques comprise: detection based upon assays that recognize that sera with arNOX exhibits a higher rate of cytochrome c reduction than sera without arNOX; an assay which measures the disappearance of the ascorbate radical spectrophotometrically by measuring the absorbance at about 265 nm since arNOX reduces an electron acceptor, e.g., ascorbate radical; by measuring the reduction of NADH by arNOX using methods known in the art; assays based on the unique oscillation property of arNOX; arNOX may be detected by resistance to retinoic acid, since NOX from healthy cells is inhibited by retinoic acid and arNOX is not inhibited by retinoic acid; a method using arNOX to identify cells where mitochondrial functions are depressed and consequently, PMOR is overexpressed; and cells may be identified in the presence of overexpressed arNOX (Morre, 1998, Plasma Membrane Redox Systems and their Role in Biological Stress and Disease 121-156; Morre et al., 1999, Mol. Cell. Biochem. 200:7-13, wherein each of the referenced documents is incorporated by reference in its entirety).

5. Methods of Identifying Agents that Interact with arNOX

The present invention relates to in vitro and in vivo methods for screening for agents which target arNOX. Within the broad category of in vitro selection methods, several types of methods are likely to be particularly convenient and/or useful for screening test agents comprising: methods which measure a binding interaction between two or more components; and methods which measure the activity of an enzyme which is one of the interacting components, i.e., arNOX. See, for example, the description in Pub. App. No. WO 00/57871, the disclosure of which is incorporated herein by reference.

Binding interactions between two or more components can be measured in a variety of ways known in the art. One approach is to label one of the components with an easily detectable label, place it together with the other component(s) in conditions under which they would normally interact (e.g., ubiquinone), perform a separation step which separates bound labeled component from unbound labeled component, and then measure the amount of bound component. The test agent may be labeled with a various detectable markers, and the he separation step in this type of approach can be accomplished in various ways. See, for example, Pub. App. No. WO 00/57871.

The invention also comprises in vitro selection method which may be used is the screening of combinatorial chemistry libraries using ubiquinone, ubiquinone derivatives, plant extracts, dormin, IBR-DORMIN®, or processed Narcissus tazzeta products as a base molecule (U.S. Pat. No. 5,565,324, which is incorporated by reference in its entirety), in vivo screening methods, gene therapy approaches (U.S. Pat. No. 5,093,246, which is incorporated by reference in its entirety) and yeast two-hybrid assays to identify test agents that interact with arNOX (Fields and Song, 1989, Nature 340:245-246, which is incorporated by reference in its entirety). The invention further encompasses methods for monitoring patient response to the agents described in this invention.

6. Target Disorders

Disorders that can be treated by the methods of the present invention include any clinical condition in which oxidative species have been implicated. Examples of clinical conditions in which oxidative species have been implicated include, but are not limited to, ischemia-reperfusion injury (e.g., stroke/myocardial infarction and organ transplantation), cancer, aging, arthritis associated with age, fatigue associated with age, alcoholism, red blood cell defects (e.g., favism, malaria, sickle cell anemia, Fanconi's anemia, and protoporphyrin photo-oxidation), iron overload (e.g., nutritional deficiencies, Kwashiorkor, thalassemia, dietary iron overload, idiopathic hemochromatosis), kidney (e.g., metal ion-mediated nephrotoxicity, aminoglycoside nephrotoxicity, and autoimmune nephrotic syndromes), gastrointestinal tract (e.g., oral iron poisoning, endotoxin liver injury, free fatty acid-induced pancreatitis, nonsteroidal anti-inflammatory drug induced gastrointestinal tract lesions, and diabetogenic actions of alloxan), inflammatory-immune injury (e.g., rheumatoid arthritis, glomerulonephritis, autoimmune diseases, vasculitis, and hepatitis B virus), brain (e.g., Parkinson's disease, neurotoxins, allergic encephalomyclitis, potentiation of traumatic injury, hypertensive cerebrovascular injury, and vitamin E deficiency), heart and cardiovascular system (e.g, atherosclerosis, adriamycin cardiotoxicity, Keshan disease (selenium deficiency) and alcohol cardiomyopathy, eye (e.g, photic retinopathy, occular hemorrhage, cataractogenesis, and degenerative retinal damage), amyotrophic lateral sclerosis, and age-related macular degeneration (Slater, 1989, Free Rad. Res. Comm. 7:119-390; Deng et al., 1993, Science 261:1047-1051; Seddon et al., 1994, JAMA 272:1413-1420; Brown, 1995, Cell 80:687-692; and Jenner, 1991, Acta Neurol. Seand. 84:6-15).

The invention is also directed to preventing or alleviating complications of diabetes, atherogenesis, atherosclerosis, and related diseases. Oxidative stress and LDL oxidation are common complicating features in diabetics and circulating AR-NOX offers opportunities for redox modulation of blood constituents important to aging, atherogenesis, and atherosclerosis (Kennedy and Lyons, 1998, Metabolism 56;14-21).

In one embodiment, the invention is directed towards a method of preventing a complication of a primary disorder in patients wherein said complication results from oxidative damage resulting from the generation of reactive oxygen species by arNOX. The method comprises administering. to a patient with a primary disorder, in an amount effective to prevent said complication, an agent or agents that sequesters arNOX, in a pharmaceutically acceptable carrier.

In another embodiment, the invention is directed towards a method of preventing a secondary disorder in patients having a primary disorder that causes oxidative damage resulting from the generation of reactive oxygen species by arNOX. The method comprises administering to a patient having a primary disorder, in an amount effective to prevent said secondary disorder, an agent or agents that sequesters arNOX, in a pharmaceutically acceptable carrier.

One embodiment of the invention provides agents and method of using said agents to ameliorate and prevent dermatological disorders comprising: Acne Vulgaris, Adiposis Dolorosa, Albinism, Alopecia, alpha 1-Antitrypsin Deficiency, Baldness, Behcet's Syndrome, Birthmarks, Birt-Hogg-Dube Syndrome (not on MeSH), Blister, Cafe-au-Lait Spots, Cellulitis, Cholesteatoma, Connective Tissue Diseases, Contractural Arachnodactyly, Congenital (Beal's Syndrome) (not on MeSH), Cutis Laxa, Decubitus Ulcer, Dercum Disease, Dermatitis, Dermatitis Exfoliative, Dermatitis Herpetiformis, Ectodermal Dysplasia, Eczema, Ehlers-Danlos Syndrome, Epidermolysis Bullosa, Erysipelas, Erythema Multiforme, Exanthema Subitum, Furunculosis, Granuloma Annulare, Gustatory Sweating, Hailey-Hailey Disease, Hair Diseases, Hair Loss, Head Lice, Hidradenitis Suppurativa, Hirsutism, Hives, Hypohidrosis, Ichthyosis, Immersion Foot, Incontinentia Pigmenti, Keloid, Keratosis Actinic (not on MeSH), Keratosis Follicularis, Keratosis Seborrheic, Leg Ulcer, Lentigo, Lichen Planus, Lichen Sclerosus et Atrophicus, Lipodystrophy, Lupus, Lupus Erythematosus Cutaneous, Lupus Erythematosus Systemic, Marfan Syndrome, Mastocytosis, Melanoma, Melanosis, Mixed Connective Tissue Disease, Nail Patella Syndrome, Nail Diseases, Nails Ingrown, Panniculitis, Parapsoriasis, Paronychia, Pemphigoid Bullous, Pemphigus, Pemphigus Benign Familial, Photosensitivity Disorders, Pigmentation Disorders, Pityriasis, Poison Ivy, Port-Wine Stain, Pruritus, Pseudoxanthoma Elasticum, Psoriasis, Pyoderma Gangrenosum, Rosacea, Scabies, Scleroderma, Scleroderma Systemic, Seborrheic Dermatitis, Shopping, Skin Cancer, Skin and Connective Tissue Diseases, Skin Diseases, Skin Diseases Infectious, Skin Ulcer, Stevens-Johnson Syndrome, Stickler Syndrome (not on MeSH), Sweat Gland Diseases, Sweet's Syndrome, Swimmer's Itch, Tinea Versicolor, Urticaria, Vitiligo, Warts, Xanthomatosis, Xeroderma Pigmentosum

7. Processed Narcissus tazzeta Products

One embodiment of the invention comprises treating patients with pharmaceutically active amount of processed Narcissus tazzeta products. A preferred embodiment of the processed product is IBR-DORMIN®. IBR-DORMIN® is comprised of a water extract of Narcissus tazzeta bulbs, and therefore soluble in water. The extraction process, such as extraction, precipitation and filtration eliminates some of the bulb material as well as part of the water. IBR-DORMIN® preferably is comprised of: water, at least one Narcissus tazzeta extract and at least one preservative. Phenochem, a blend of paraben esters and phenoxyethanol, is an example of a preferred preservative. Narcissus tazzeta extracts may be present in various amounts in agents used to treat mammals. For example processed Narcissus tazzeta products may be present in amounts measured by percentage of total volume: between 25-49.9%, between 10-24.9%, between 5-9.9%, between 1-4.9%, between 0.1-0.99%, and less than 0.1%. Additional information about IBR-DORMIN® can be found in U.S. Pat. Nos. 6,635,287 and 6,347,254, the disclosure of which is also incorporated herein by reference.

A feature of processed Narcissus tazzeta products are their ability to slow cell proliferation. Processed Narcissus tazzeta products can induce reversible dormancy in other plants. Processed Narcissus tazzeta products have also shown inhibitory effects on cell growth of human fibroblasts and keratinocytes primary cultures as well as on cancerous strains. This effect is thought to take place through a slowdown of the cell cycle in phase S, G2 and M, as FACS studies have shown, resulting in a decrease of the cell pool in G1.

One embodiment of the invention is the utilization of agents comprised of processed Narcissus tazzeta products to produce cutaneous antagonism between growth and differentiation (e.g., psoriasis). For example an agent comprised of IBR-DORMIN® in the form of a cream could be used to treat psoriasis. Various concentrations of IBR-DORMIN® may be utilized to affect desired efficacy of treatment.

In theory, processed Narcissus tazzeta products may be used wherever slowing cell proliferation is a benefit, such as: reduction the rate of nail growth, prolonging sun tan, treatment of skin disorders including acne, treatment of psoriasis, hair removal treatments, inhibition of alopecia and hirsutism, decrease in pigmentation, treatment for people with high risk for benign or malignant tumor.

One of the identified active fractions in processed Narcissus tazzeta products is at molecular size less than 5,000 Dalton. The extraction process therefore preferably includes an ultra-filtration step through a 5,000 D cut-off membrane. This active fraction is heat stable. It can be autoclaved at 120 C, 2 atmospheres for 30 min. and retain 99% of initial activity. The extract or agents containing the extract should preferably be kept sterile, in closed containers at 4 to 24° C. The inhibiting activity of the extracts processed according to this invention is stable at room temperature for two years with no loss of activity.

8. Inhibition of arNOX by Narcissus tazzeta Products

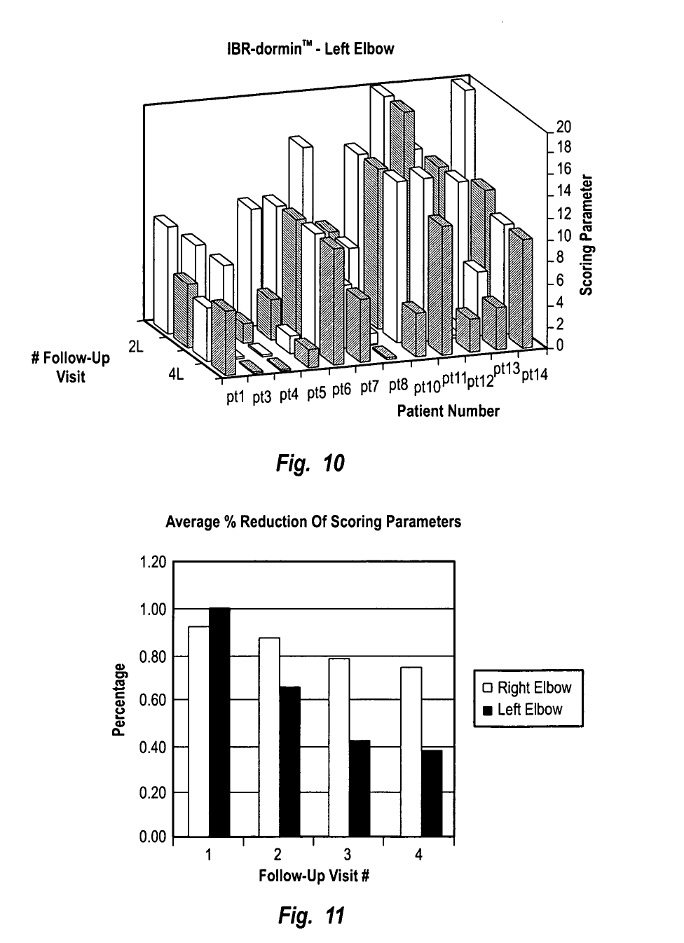

Processed Narcissus tazzeta products sequester arNOX activity. The inhibition of arNOX results in a decrease in the generation of reactive oxygen species by arNOX. A decrease in reactive oxygen species results in a decrease of oxidative damage resulting from said reactive oxygen species. For example, IBR-DORMIN® is a complex mixture from dormant Narcissus tazzeta bulbs for which anti-aging activity is claimed. The preparation specifically and completely inhibits the arNOX activity of sera and of transfusion buffy coats (FIG. 10). The invention encompasses the use of IBR-DORMIN® for inhibition of arNOX. (FIG. 10). As such, the processed Narcissus tazzeta product preparations may be utilized as disclosed herein to ameliorate conditions associated with a variety of aliments.

One embodiment of the invention comprises the use of agents comprising processed Narcissus tazzeta products, IBR-DORMIN®, and/or coenzyme Q, alone or in combination with each other for inhibition of arNOX.

Another embodiment of the invention further comprises the use of inhibition agents other than processed Narcissus tazzeta products, IBR-DORMIN® and coenzyme Q such as Shisandra Chinensis, Lonicera Japonica, Fagopyrum Cymosum and methylparaben.

The pharmaceutical compositions of this invention may comprise varying modes of administration of compounds that sequester arNOX. The modes of administration of compounds comprise capsules, tablets, soft gels, solutions, suppositories, injections, aerosols, or a kit.

Embodiments of the invention comprises the isolation and characterization of arNOX using processed Narcissus tazzeta products, preferably IBR-DORMIN® as an inhibition agent.

The invention contemplates the isolation and purification of arNOX, cloning of the arNOX cDNA and a complete molecular characterization of the arNOX protein.

Existing assays will be employed to fractionate processed Narcissus tazzeta products to identify the active constituent(s) and to assay other natural product sources for anti-aging activities. Based on the molecular information, a rapid and non-invasive RT-PCR (reverse transcriptase-polymerase chain reaction) skin test for arNOX expression will be developed along with models to test the necessity and/or sufficiency of arNOX in the aging process.

The invention encompasses the use of topical administration of processed Narcissus tazzeta products to, maintain skin vitality and for the oral administration of coenzyme Q as an approach to ablation of age-related cell surface and lipoprotein oxidation. A preferred embodiment of the invention comprises the topical administration of a cream, which comprises IBR-DORMIN®, to the skin of patients to maintain and improve skin vitality.

One embodiment of the invention comprises therapeutic agents and the administration of a therapeutically effective amount of a formulation comprised of at least one therapeutic agent. One embodiment of the therapeutic agents of this invention comprises at least one processed Narcissus tazzeta product. The agent may further comprise ubiquinones. The formulation may be administered to a patient with a disorder or a complication of a disorder caused by oxidative damage resulting from the generation of reactive oxygen species. For example the formulation may be administered to a patient with a disorder or a complication of a disorder caused by oxidative damage resulting from the generation of reactive oxygen species by arNOX. In a preferred embodiment, the total daily amount of the therapeutic agent administered is from about 1 to about 500 mg. In a more preferred embodiment, the total daily amount administered is from about 1 to 100 mg of therapeutic agent.

In one embodiment, the invention is used to identify patients suffering from disorders associated with reactive oxygen species who may be responsive to treatment with the therapeutic agents disclosed in this invention. Such responsive patients may be identified by assay of serum or urine for superoxide generation, which is responsive to treatment comprising the therapeutic agents of the present invention. The generation of superoxide may be followed by reduction of cytochrome c or any other suitable biological or chemical method.

In one embodiment the invention further comprises treating a patient with a pharmacologically effective amount of ubiquinones to inhibit the generation of reactive oxygen species. In a preferred embodiment, the ubiquinones are of the human derivative Q₁₀. In another embodiment, the ubiquinones comprise the naturally occurring derivatives Q₆, Q₇, Q₈, and Q₉. In another embodiment, the ubiquinones comprise other derivatives Q₁, Q₂, Q₃, Q₄, Q₅, Q₁₁, and Q₁₂. In another embodiment, the invention comprises mixtures of the ubiquinone derivatives described supra. The invention further comprises all pharmaceutically acceptable derivatives of the compositions listed supra for methods of treating a patient with an arNOX related disorder, with ubiquinone administration in the range of 0.1 to 100 mg per kg body weight.

The invention also encompasses methods for monitoring patient response to the agents of the present invention. Preferably the patients would be monitored for responsiveness to to treatments comprising the administration of processed Narcissus tazzeta products, and which may further comprise the administration of ubiquinones. By monitoring circulating arNOX activity in patient sera, it will be possible to determine therapeutic dosages and to monitor therapeutic benefit from the therapeutic agents of the invention. The response to the subject compositions may be monitored by assaying the blood or urine of the patient for the arNOX activity that is responsive to the compositions of this invention. By following the above monitoring procedures, an effective dosage of the subject compositions may be administered in accordance with the requirement of the individual patient.

9. Pharmaceutical Formulations

Agents that interact with arNOX identified in this invention may be formulated into pharmaceutical preparations for administration to mammals for prevention or treatment of disorders in which oxidative species have been implicated. In a preferred embodiment, the mammal is a human. Compositions comprising a compound of the invention formulated in a compatible pharmaceutical carrier may be prepared, packaged, and labeled for treatment. If the complex is water-soluble, then it may be formulated in an appropriate buffer, for example, phosphate buffered saline or other physiologically compatible solutions.

Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parentenal, rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-hydroxybenzoates or sorbic acid). The pharmaceutical compositions may take the form of for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, tale or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound. For buccal administration, the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, diehlorotetrafluoroethane, carbon dioxide or other suitable gas. In the ease of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. The compounds may also be formulated as a topical application, such as a cream or lotion.

In addition to the formulations described previously, the compounds may also be formulated as a depot preparation. Such long acting formulations may be administered by implantation (for example, subcutaneously or intramuscularly) or by intramuscular injection.

Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The composition may be formulated as compositions to be applied to the skin of mammals. The composition may for example be comprised of active agents and other carrier ingredients that facilitate the application of the active agent to the surface of skin. For example, the composition may be formulated as a cream or lotion for application to the skin.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the compositions in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of compositions by a clinician or by the patient.

10. Treatment of Skin

The present invention provides compositions comprising active agent(s), which prevent and/or ameliorates skin damage and associated conditions. Further, the invention encompasses methods for utilizing said compositions. The stratum corneum is the layer of the skin that forms the top surface layer and serves to protect the skin while controlling moisture and the flow of substances in and out of the skin. As this barrier function is broken down, the skin suffers damaging effects, thus creating or contributing to premature aging. These damaging effects causing premature aging of the skin are a concern for many individuals wishing to maintain healthy, youthful looking and feeling skin. Reactive oxygen species participate in a number of destructive reactions potentially lethal to cells. Reactive oxygen species are responsible in part for deleterious cellular interactions including impairing fibroblast cells ability to produce healthy collagen and elastin. Furthermore, the skin is subject to deterioration through dermatological disorders, environmental abuse (wind, air conditioning, central heating) or through the normal aging process (chronoaging), which may be accelerated by exposure of skin to sun (photoaging).

A preferred embodiment of the invention provides active agents from processed plants for the treatment of skin. The active agents prevent and/or ameliorate skin damage and associated conditions. In one embodiment of the invention the processed plant products sequester arNOX activity. In another embodiment of the invention, the processed plant products inhibit radical oxygen species. In another embodiment agents and methods of the invention prevent and/or improve the health of the skin. For example, the agents may improve skin tone, and helps diminish the appearance of fine lines and visible signs of aging. In another embodiment of the invention, the agents positively affects the body's natural production of collagen and elastin. In another embodiment, the agents of the invention minimize the effects of environmental agitators such as pollution, sun, free radicals and stress.

One embodiment of the invention provides compositions, and methods for using the same, for preventing and/or ameliorating dermatological disorders and the effects thereof.

One embodiment of the invention provides composition for preventing and reducing the effects of the production of reactive oxygen species and methods for using the same. For example, the invention encompasses the use of active agents derived from plants to sequester arNOX activity. Further, the invention contemplates the use of other synthetic and natural compounds to sequester arNOX activity.

The present invention discloses compositions, which treat the skin and delays the visible signs of actual aging and weathered skin such as wrinkles, lines, sagging, hyperpigmentation and age spots. The present invention also decreases the appearance and condition of sensitive, dry and/or flaky skin, serves to soothe red, and/or irritated skin, and treats spots, pimples, blemishes, and other skin irregularities.

The present invention advances prior art compositions by providing compositions and methods for using the same not previously disclosed. The invention provides pharmaceutical compositions, methods of use, and pharmaceutical kits for the treatment of disorders resulting from oxidative changes in cells that result in aging by targeting an aging-related isoform of NADH oxidase (arNOX), shed into the sera by aging cells. The compositions may contain agents extracted from plants. For example, the compositions of the invention may comprise at least one processed Narcissus tazzeta product, whether alone or with other inhibition agents and inhibit the activity of an aging-related isoform of NADH oxidase shed into the sera by aging cells. The composition may comprise ubiquinones, extracts of Shisandra Chinensis, Lonicera Japonica, Fagopyrum Cymosum, methlyparaben, L-Carnosine, Propylparaben, Ethylparaben, L-Ergothioneine, Betulinic acid, Solanum Lycopersicum, Univestin, Soliprin., coenzyme Q₁₀, and/or preservatives. In a preferred embodiment the processed Narcissus tazzeta extract is IBR-DORMIN®. The active agent(s) may be incorporated into various carriers suitable for application to the skin. Additional elements such as colorants, fragrances, and other ingredients, such as skin protectants, may also be present.

In one embodiement a portion of, or all of these ingredients may be combined with other ingredients commonly found in anti-aging and repair serum formulations. Vehicles, other than, or in addition to water can include liquid or solid emollients, solvents, humectants, thickeners and powders. The vehicle may be from 0.1% to 99.9%, preferably from 25% to 80% by weight of the composition, and can, in the absence of other cosmetic adjuncts, form the balance of the composition. In one embodiment, the vehicle is at least 80% water, by weight of the vehicle. In another embodiment water comprises at between about 50% to 85% of the composition by weight. In yet another embodiment, water is present between about 0.1% to 55%, by weight of the composition. In other embodiments other vehicles are used in the above recited concentrations.

An oil or oily material may be present, together with an emulsifier to provide either a water-in-oil emulsion or an oil-in-water emulsion, depending largely on the average hydrophilic-lipophilic balance (HLB) of the emulsifier employed.

The inventive compositions may also include sunscreens. Sunscreens include those materials commonly employed to block ultraviolet light. Illustrative compounds are the derivatives of PABA, cinnamate and salicylate. For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the emulsions can vary depending upon the degree of protection desired from the sun's UV radiation.

Emollients may further be incorporated into cosmetic compositions of the present invention. Levels of such emollients may range from 0.5% to 50%, preferably between 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols, which may serve as emollients are linear and branched chain alkyl polyhydroxyl compounds. For example, propylene glycol, sorbitol and glycerin are preferred. Also useful may be polymeric polyols such as poly-pronylene glycol and polyethylene glycol. Butylene and propylene glycol are also especially preferred as penetration enhancers.

Exemplary hydrocarbons which may serve as emollients are those having hydrocarbon chains anywhere from 12 to 30 carton atoms. Specific examples include mineral oil, petroleum jelly, squalene and isoparaffins.

Other embodiments of the compositions of the present invention comprise thickeners. A thickener will usually be present in amounts anywhere from 0.1 to 20% by weight, preferably from about 0.5% to 10% by weight of the composition. Exemplary thickeners are cross-linked polyacrylate materials available under the trademark CARBOPOL®® from the B.F. Goodrich Co. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range anywhere from 0.001% up to 20% by weight of the composition.

The composition of the invention may be used for topical application to human skin, as an agent for conditioning, moisturizing and smoothing the skin, increasing the flexibility and elasticity and preventing or reducing the appearance of wrinkled, lined or aged skin. The unique formulation of the present invention offers the complete response to the loss of skin tone and promotes immediate and continuous benefits to effectively boost hydration and firmness of the surface layer of the skin, all while working to repair the underlying layers of the skin with antioxidants and other beneficial ingredients to help diminish the appearance of fine lines and wrinkles and to restore visible tone and elasticity.

In one embodiment a small quantity of the composition comprised of from about 1 to 1000 ml of active agent, is applied to the skin. In a preferred embodiment, a quantity of composition comprising from about 1 to 100 ml of active agent is applied to the skin. This process may be repeated several times daily for any period of time. Preferably, the composition is applied to the skin once in the morning and once in the evening.

The topical skin care composition of the invention can be formulated as a lotion, a cream or a gel. The composition can be packaged in a suitable container to suit its viscosity and intended use by the consumer. For example, a lotion or a cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

### EXAMPLE 1

### Characterization of arNOX

1. Superoxide Production By Buffy Coats

Reduction of ferric cytochrome c by superoxide was employed as a standard measure of superoxide formation (Mayo, L. A. and Cumutte, J. (1990) Meth. Enzyme. 186, 567-575. 7. Butler, J, Koppenol, W. H. and Margollash, E. (1982) J. Biol. Chem. 257, 10747). If superoxide dismutase was added to remove the superoxide as it was generated, the reduction of ferric cytochrome c was prevented to confirm that ferric cytochrome c reduction in the assay was due to superoxide.

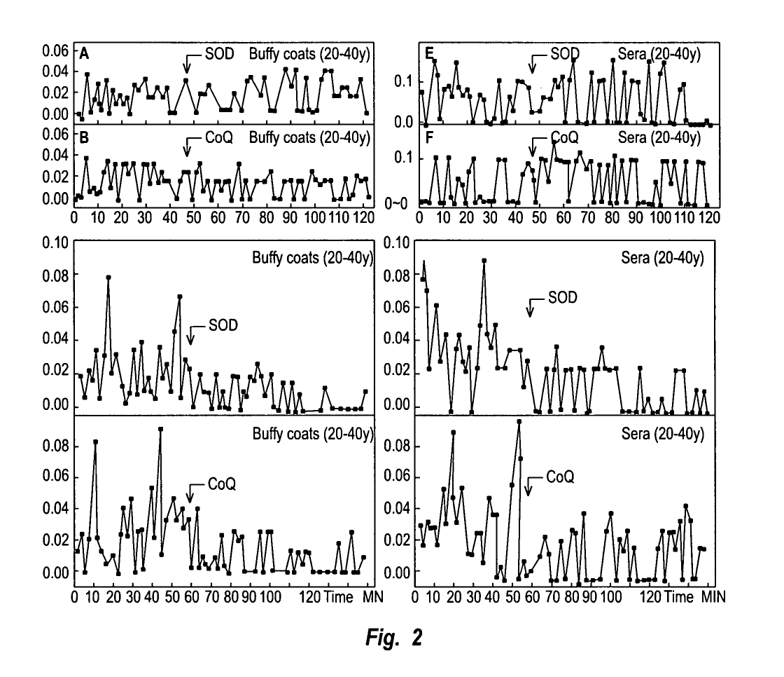

Buffy coats were pooled from aged individuals (70-100 y) and the reduction of ferric cytochrome c was observed (FIG. 2) with an oscillating activity. The oscillations exhibited a period length of ca. 25 min (arrows, FIGS. 2C and 2D). This oscillatory reduction of cytochrome c was absent from buffy coat fractions from younger (20-40 y) individuals (FIGS. 2A and 2B). The oscillating reduction of ferric cytochrome c was inhibited completely by superoxide dismutase (SOD) (FIG. 2C) and by 100 μM coenzyme Q (EC₅₀ of 20 μM) (FIG. 2D). The rate of coenzyme Q inhibited ferric cytochrome c reduction was 7-fold greater in buffy coat fractions of 90-94 y individuals as compared to 80-89 y individuals (Table 1). Buffy coats of less than 65 y individuals lacked the activity.

2. Superoxide Production and Inhibition By Coenzyme Q and Superoxide Dismutase

Assays of ferric cytochrome c reduction in sera compared 53 samples from young (20 to 40 y) and 65 samples from aged (80 to 100) individuals. Activities were 0.2±0.2 nmoles/min/100 μl sera for young compared to 1.4±0.2 nanomoles/min/100 μl sera for aged. With untreated serum samples, addition of 30 units/ml of superoxide dismutase inhibited the activity by 40±10%. Addition of 300 μg/ml coenzyme Q also inhibited the activity by 40% although on average the results were more variable.

When SOD (30 units) and coenzyme Q (450 μg) were added sequentially to the same reaction mixture and assayed over 300 sec, no further inhibition to the rate of ferric cytochrome c reduction in serum was observed when coenzyme Q was added after maximal SOD inhibition and vice versa.

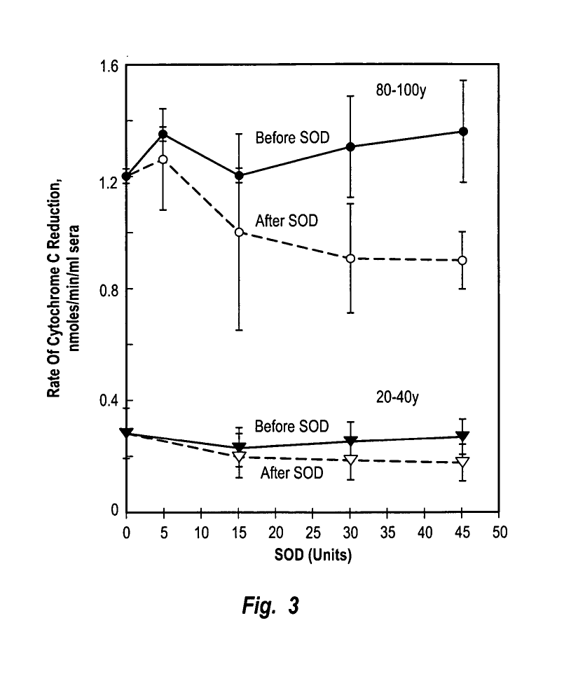

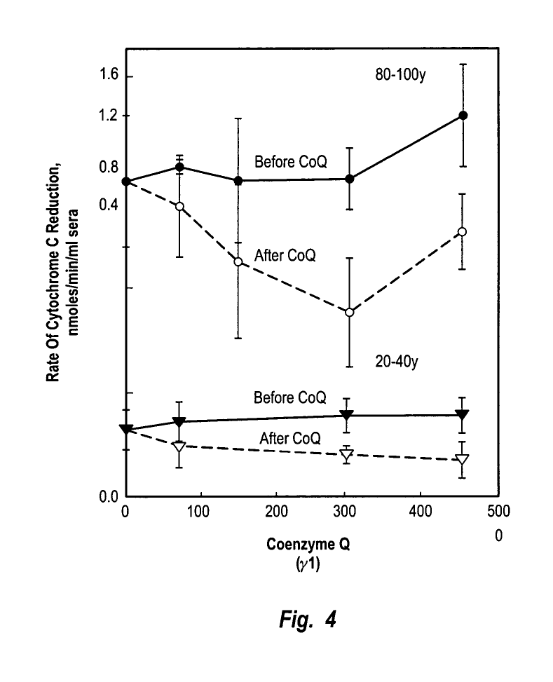

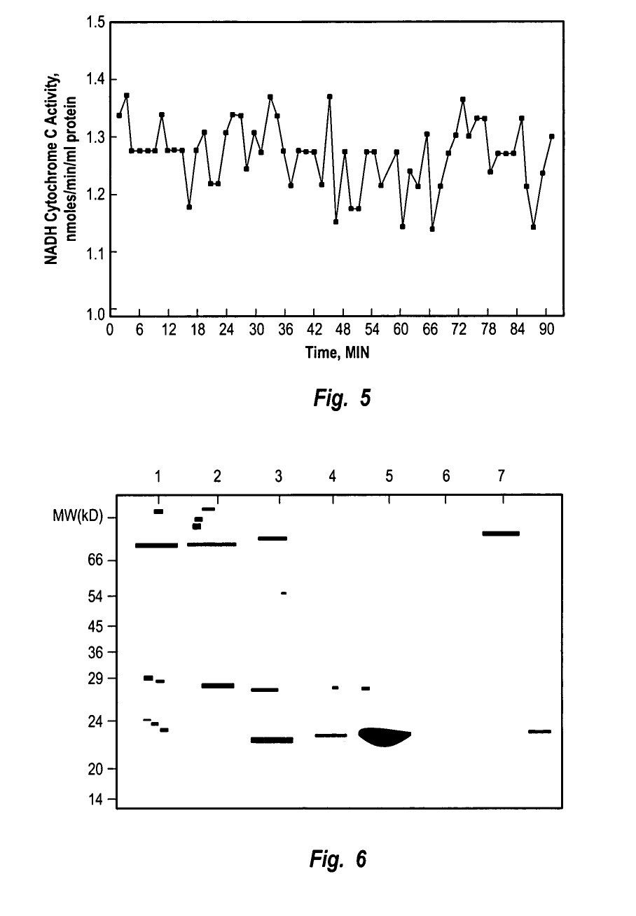

Inhibition of the rate of age-related cytochrome c reduction (ACR) was in proportion to the SOD concentration between 8 and 35 units (FIG. 3). A plateau was reached at 45 units. With coenzyme Q, inhibition was proportional to amounts between 75 μg to 450 μg and reached a plateau at about 450 μg coenzyme Q (FIG. 4).

Additional reduction of cytochrome c by sera was observed in the presence of NADH (NADH-cytochrome c reductase). However, the NADH-stimulated activity was about 4 nmoles/min/ml of sera for sera of both young and aged individuals. The aging-specific increment of ca. 2 nmoles/min/mg protein was observed both in the presence or absence of NADH. Neither SOD nor coenzyme Q inhibited the activity of NADH cytochrome c reductase in serum of either young or aged individuals. Also, the addition of coenzyme Q did not significantly inhibit the activity of authentic NADH cytochrome c reductase of pig liver microsomes.

Thus the arNOX appears to be unrelated to NADH-cytochrome c reductase. Composed of a large hydrophilic, catalytic domain and a smaller hydrophobic membrane binding segment, proteases release the active protein from, membranes. The NH₂ terminal glycyl residue is linked to the membrane via myristic aid. Solubilization can be achieved by enzymatic digestion without loss of enzymatic activity. Lysosomal acid proteases, i.e., capsaicin D, also release the activity. Also, as arNOX does not respond to capsaicin or (−)-epigallocatechin gallate (EGCg), it is not one of the drug-responsive tNOX isoforms.

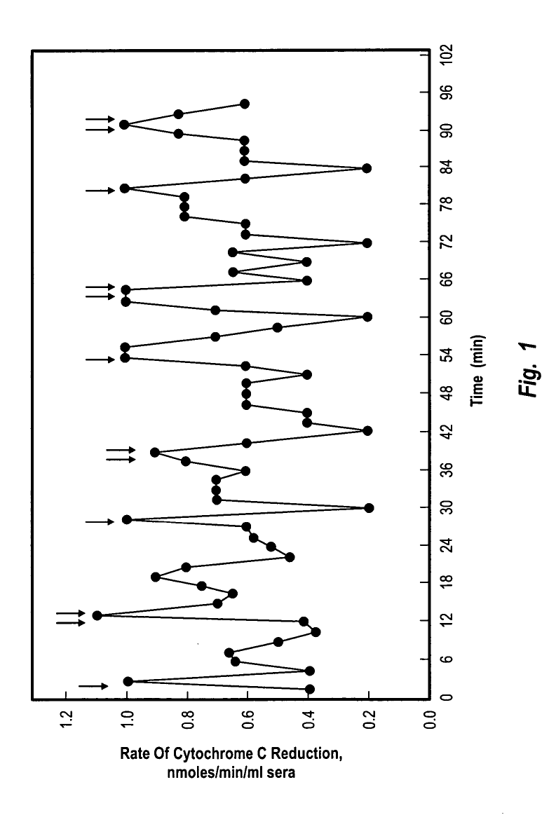

An oscillating rate of enzymatic activity with a regular period length of about 24 min is one of the defining characteristic of the CLOX family of proteins. When the reduction of ferric cytochrome c of individual sera of 90-100 y subjects was assayed over 1 min at intervals of 1.5 mm, the activity was observed to oscillate with a regular period length but again with a period length of 25 min rather than 24 min (FIG. 1). The oxidation of NADH measured in parallel with the same sample showed two patterns of oscillations, one with a period length of ca. 25 min corresponding to the age-related isoform and a second pattern with a period length of 24 min corresponding to CNOX as reported previously. Corresponding oscillations were not observed with the activity of NADH cytochrome c reductase of pig liver microsomes as a positive control (FIG. 6).

TABLE 1
Reduction of Ferricytochrome c. Measure of Superoxide
Production by Buffy Coats from Blood of Aged Individuals
and Inhibition by Coenzyme Q.

Rate of reduction
of ferricytochrome c
nmoles min⁻¹ 10⁷ cells⁻¹)

| Group | N | No addition | +100 μM Q₁₀ |
|---|---|---|---|
| 35-65 years | 5 | ND | ND |
| 80-89 years | 6 | 0.25 ± 0.02 | −0.03 ± 0.02 |
| 90-94 years | 6 | 0.36 ± 0.07 | −0.07 ± 0.07 |

Values are means ± standard deviation;
ND, not detected
Q₁₀ ubiquinone-10 CoQ₁₀).
In these experiments, the negative rates reflect small negative slopes in the rate of NADH oxidation. Statistically the rates were zero.

TABLE 2
Response of Rate of Reduction of Ferricytochrome c of Pooled Serum
Samples to Proteinase K Digestion.

Rate of reduction
of ferricytochrome c
nmoles m⁻¹ mi serum⁻¹)

| Group | | N | No addition | +Proteinase K |
|---|---|---|---|---|
| <35 y | females | 12 | 0.24 ± 0.14 | 0.26 ± 0.17 |
| 35-45 | males | 9 | 0.21 ± 0.15 | 0.27 ± 0.16 |
| 75-85 | males | 10 | 0.7 ± 0.35 | 0.72 ± 0.14 |
| 75-85 | females | 8 | 1.1 ± 0.20 | 1.0 ± 0.18 |
| >90 | females | 10 | 0.8 ± 0.11 | 0.94 ± 0.24 |

N = Number of subjects represented in each pooled sample.

TABLE 3
Superoxide Production (Reduction of Ferricytochrome c) in
Sera of Aged (80-98 y) individuals Following Treatment with
Proteinase K and Response to Superoxide Dismutase (n = 10).

Nmoles/min/mi/sera

| Group | N | No addition |
|---|---|---|
| No proteinase K | 0.7 ± 0.2 | 0.42 ± 0.1 |
| After proteinase K | 0.6 ± 0.2 | 0.22 ± 0.1 |

Source of electrons for cytochrome c reduction with sera of aged individuals. The regular pattern of oscillations with a period length of 25 min that correlates with a corresponding pattern of oscillations for NADH oxidation dictates that the source of electrons for the oscillating generation of superoxide reduction of tonic cytochrome c for buffy coats and in the sera of aged patients is the age-related NOX protein. The regular period length of ca 25 min distinguished the activity from that of other proteins including the constitutive CNOX protein of sera which has a period length of 24 min and does not generate superoxide (i.e., reduce ferric cytochrome C). Within the age-related NOX protein, active site cysteines and bound copper were considered as electron sources. The serum activity was unaffected by the copper chelators bathocuproene or bathocuproenedisulforiate. A protein thiol source was considered more likely since the activity was inhibited by thiol reagents such as p-chloromercuribenzoate.

The serum source to regenerate the NOX protein thiols oxidized during the reduction of cytochrome c also appears to be protein thiols. NOX proteins exhibit protein disulfide-thiol interchange activity and are capable of undergoing protein thiol oxidation and protein disulfide reduction at the expense of external protein sources. Copper as a serum source of electrons is less likely since added copper did not enhance the activity nor did the copper chelators inhibit.

Serum levels of protein thiols are certainly adequate to fuel the reaction. By direct assay using Ellman's reagent, the sera analyzed were calculated to contain sufficient thiols to sustain the average rate of cytochrome c reduction for more than 10 days at room temperature and for several months in the cold assuming that all, thiols were available for reaction. Serum samples where protein SH was oxidized with dilute (0.01%) hydrogen peroxide followed by catalase to remove residual hydrogen peroxide were inactive. Catalase by itself was without effect. Also, oxidation of proteins by equilibration with low concentrations of GSSG inactivates serum activity but not that of buffy coats where the source of electrons is assumed to be from the electron transport pathway. Addition of GSH neither stimulates nor inhibits but may eventually prolong the cytochrome c reduction capacity of the sera by maintaining levels of protein thiols. The lack of complete inhibition by SOD or coenzyme Q of FIGS. 3 and 4 results in large measure from a basal activity that is insensitive to inhibition. As shown by data of FIG. 2, the oscillating activity component in sera of aged patients is eliminated by both SOD (FIG. 2G) and coenzyme Q (FIG. 2H). The source of the basal activity appears to involve neither a specific enzyme nor cytochrome c reduction. It is encountered in other serum NOX assays and appears to result from light scattering changes due to aggregation of serum proteins.

3. Proteinase K Digestion.

Resistance to proteinase K digestion is a very important characteristic of the CLOX proteins. There was no significant decrease in the rate of arNOX following proteinase K digestion (Table 2). Inhibition by superoxide dismutase increased from 40% to 60% following proteinase K digestion of sera from aged individuals (Table 3), due to a marked reduction in the basal absorbance changes attributed to protein aggregation. The protein thiol content of the sera was not affected by proteinase digestion.

4. Western Blotting for arNOX

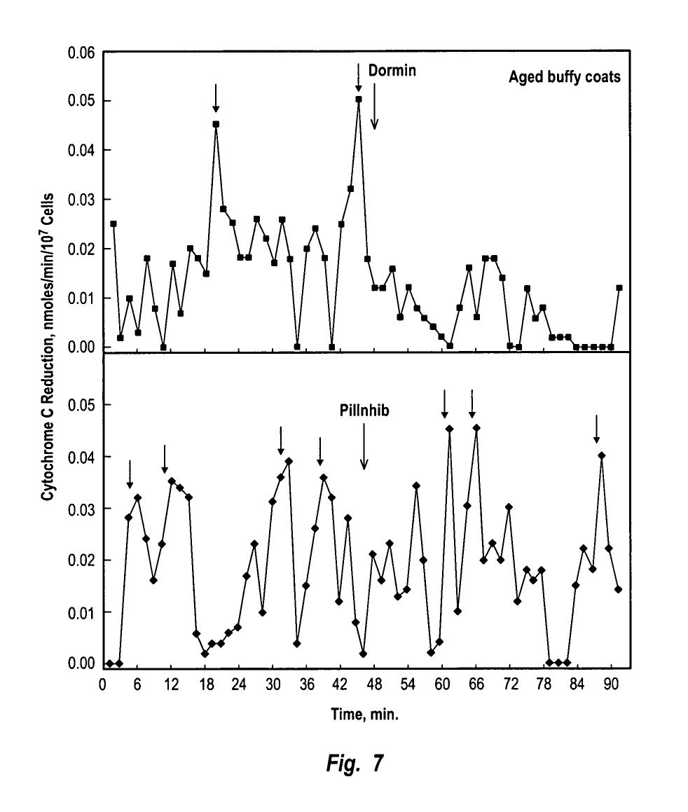

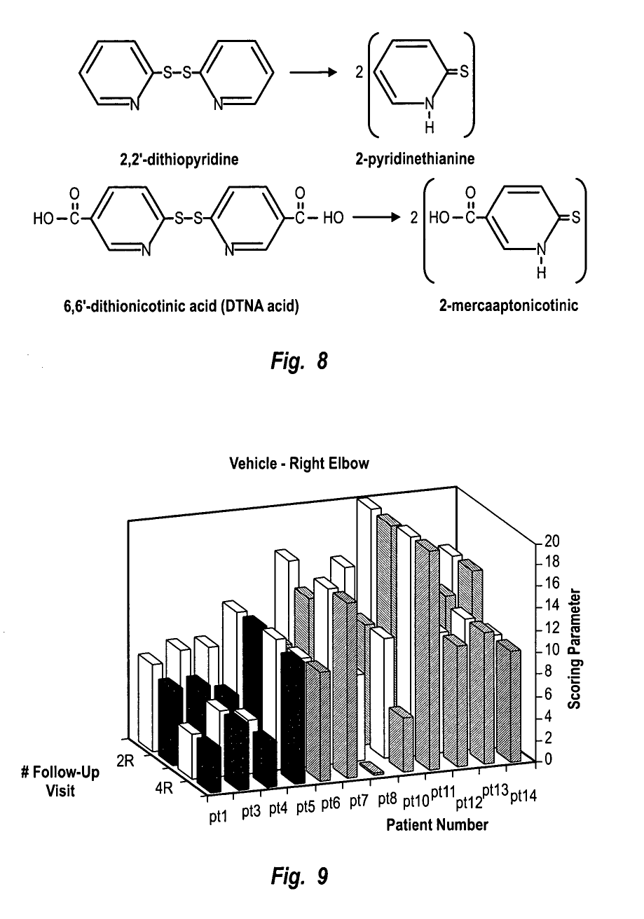

To further verify that the arNOX is the result of a NOX protein of the CLOX protein family, polyclonal anti-sera to the C terminus of a previously-cloned and tumor-specific NADH oxidase (tNOX) were utilized to identify an immunoreactive band on Western blots. Serum samples, after proteinase K digestion to reduce background proteins, were separated by 10% SDS-PAGE and transferred by electroblotting onto nitrocellulose membranes. A reactive band at a molecular weight of ca. 22 kD was observed on lanes from samples of sera of elderly subjects (FIG. 6). There was a strong correlation between band intensity determined by densitometry and arNOX (FIG. 7). A similar correlation was seen between band density and the age of the subject (FIG. 8). To verify that the 22 kD region of the gel contained a CLOX protein, an SDS-PAGE gel was cut into segments and the proteins were eluted. Activity was restored by reduction of the protein with 100 μM NADH, pH 7.0, followed by addition of 0.03° h hydrogen peroxide to reoxidize the refolded protein. The region of the gel corresponding to a molecular weight of 22 kD exhibited an oscillating pattern of ferric cytochrome c reduction (FIG. 9). The remainder of the gel slices lacked a reproducible pattern of ferric cytochrome c reduction.

5. Purification of Protein Catalyzing Aging-Related Cytochrome a Reduction

The protein catalyzing arNOX activity was separated from serum through immunoprecipitation with (NOX antibody. The precipitated proteins were separated by SDS-PAGE and the proteins in the gel were transferred to PVDF membranes. The protein on PVDF membrane was identified by Coomassie blue staining. The target band on the PVDF membrane was excised and submitted for N-terminal amino acid sequencing.

### EXAMPLE 2

### arNOX Inhibition

Various compounds were analyzed to assess arNOX inhibition according to the methods disclosed. The compounds, product codes and names, etc. as provided in the table below (Table 4):

TABLE 4
arNOX inhibition assays

| No. | Product Code | Product Name | Lot Number | Comments |
|---|---|---|---|---|
| 1. | UP566 | Soliprin | E0404 | Free B-ring flavanoids |
| | | | | and Flavans |
| 2. | R44390 | Univestin | G1702-COX-2 | |
| 3. | 0301 | IBR-DORMIN ® | BA0303161 | Narcissus Tazzeta Bulb |
| | | | | Japanese Name: |
| | | | | Fusazakisuisen Extract |
| 4. | 0601 | IBR-TOM ® | BA t4006L | Solanum Lycopersicum |
| | | | | Japanese Name: |
| | | | | Tomato Ekisu |
| | | | | (Tomato Extract) |
| 5. | 855057 | Betulinic acid | CAS No. 472-15-1 | FW 456.71 |
| 6. | 26547 | L-Ergothioneine | 12723P | MW 229.3 |
| | | L-ERGO ™ | CAS No. | 497-30-3 |
| 7. | | Ethylparaben | | |
| 8. | | Propylparaben | | |
| 9. | | Methylparaben | | |
| 10. | C-9625-5G | L-Carnosine | CAS No. 305-84-0 | FW 226.24 (Sigma) |

The compounds listed above in Table 4 were tested initially in the standard arNOX assay at a dilution of 1:50. Solids were prepared in water at an initial concentration of approximately 100 mM and then also tested at a dilution of 1:50, i.e., 2 mM. All compounds were evaluated using human buffy coats prepared from an 86 y female. Several compounds were evaluated with sera from aged patients as follow: IRB Dormin (88 y female), IRB TOM® (88 y female), L-carnosine (88 y female, 84 y female), L-ERGO (89 y male). Serum and buffy coats gave consistent results. Compounds active at 1:50 dilutions were reassayed with buffy coats at a dilution of 1:500 and compounds active at 1:500 were reassayed with buffy coats at a dilution of 1:5000.

Details of the assay protocol are as follow. Buffy coats, a mixture of lymphocytes and platelets, were obtained from a commercial supplier. The blood samples were maintained at 4° C. prior to collection and assay. Ca. 10⁷ cells were added to each assay. Cell numbers were determined using a hemocytometer.

Measurement of arNOX activity based on ferricytochrome c reduction as a measure of superoxide production were taken. The rate of reduction of ferricytochrome c was determined from the increase in absorbance at 550 nm with 540 nm as reference. This is a widely accepted method when coupled to superoxide dismutase inhibition for the measurement of superoxide generation. The assay consists of 150 μl (2 mg/ml) of oxidized ferricytochrome c solution and 150 μl serum or 40 μl buffy coats in PBSG buffer (8.06 g NaCl, 0.2 g KCl, 0.18 g Na₂HPO₄, 0.26 g KH₂PO₄, 0.13 g CaCl₂, 0.1 g MgCl₂, 1.35 g glucose dissolved in 1000 ml deionized water, adjusted to pH 7.4, filtered and stored at 4° C.). Rates were determined using a SLM Aminco DW-2000 spectrophotometer (Milton Roy, Rochester, N.Y., USA) in the dual wave length mode of operation with continuous measurements over 1 min every 1.5 min. After 45 min, test compound was added and the reaction was continued for 45 min. A millimolar extinction coefficient of 19.1 cm⁻¹ was used for reduced ferricytochrome c. (D. M. Morré, F. Guo and D. J. Morré, 2003, Mol. Cell. Biochem. 254: 1010-109).

The following compounds were active at a dilution of 1:50 but were inactive at a dilution of 1:500: Soliprin, propylparaben and methylparaben. The buffy coats used contained two distinct arNOX activites. Methylparaben inhibited one and had no effect on the other. A similar result was seen with Soliprin. In one experiment with a serum sample containing 3 arNOX activities, L-carnosine, inhibited one arNOX, stimulated a second arNOX and was without effect on a third. The following compounds gave mixed results at a dilution of 1:50 but were inactive at a dilution of 1:500: IRB-TOM® and L-ERGO. Only the following compound was active at a dilution of 1:500: IRB-Dormin.

### EXAMPLE 3

### IBR-DORMIN® Heat Resistance

A batch of IBR-DORMIN® was produced the pH was measured as 5.84. Its color was light yellow (607c by Pantone). The batch was kept in high-density polyethylene container, at room temperature. As detailed in the table below, samples were taken to determine color, pH and activity by seeds test. Color was defined by Pantone color formula guide. pH was measured by pH meter (Radiometer, Copenhagen, Denmark). Product pH range was 4.5-6.5.

Seed test were performed as follows. Cucumber seeds were germinated over night on water-wetted filter paper in closed tray at 28° C. Seeds with 1-2 mm roots were taken for the assay. IBR-DORMIN® (x2 concentrated) was applied in the following dilutions: 50%, 25%, 12.5%, 5% and 2.5%. Tap water served as a control. 2-ml of each dilution were applied on a filter paper in a big Petri dish. Ten seeds were put in each Petri dish.

Root length was measured after 48 h. The average length of 10 seeds was calculated. A semi-logarithmic graph of root length vs. % extract was drawn. ID₅₀ (the percentage of extract required to reach 50% inhibition of root growth) was calculated from the equation of the best-fit curve. Product ID₅₀ range is 9.5-13.5%.

TABLE 5
ID₅₀ range

| PH | Color | ID₅₀ by seed test (%) |
|---|---|---|
| 5.86 | Light yellow (607c) | 9.81 |
| 5.96 | Light yellow (607c) | 10.99 |
| 5.84 | Light yellow (607c) | 10.60 |
| 5.55 | Light yellow (607c) | 11.34 |
| 5.59 | Light yellow (607c) | 10.30 |

By all the examined parameter, IBR-DORMIN® pH was found to be stable for up to 18 months. The pH and activity (ID₅₀) are within the specified range, and the color did not change.

IBR-DORMIN® was examined for its stability to heat by examining the influence of autoclaving on the liquid appearance and activity. A sample of IBR-DORMIN® was autoclaved in the lab for 30 min at 120° C. and 2 atmospheres. Three subsequent cycles of autoclave were performed. After each cycle, a portion was taken to examine maximum inhibition activity by seed test: Cucumber seeds were germinated over night on wet filter paper in closed tray at 28° C. Seeds with 1-2 mm roots were taken for the assay. IBR-DORMIN® was applied at 50%. Tap water served as a control. 2 mli of this dilution were applied on a filter paper in a Petri dish with 10 seeds. Root length was measured after 48 h. The average length of the 10 seeds was calculated, and percentages of inhibition is given by the equation:(1-(Average of dormin treated root length/Average of root length in water)).

Results of the heat stability assay were as follows. Liquid appearance: after autoclaving, small precipitates could be observed. Only a slight change in color was observed comparing non-autoclaved to autoclaved sample. Activity: The table below summarizes the inhibition activity of 50% IBR-DORMIN® on cucumber seed growth. As shown, full activity was retained after three subsequent cycles of autoclave.

TABLE 6
Heat Stability

| | Root inhibition (%) |
|---|---|
| No autoclave | 91.0 |
| First autoclave | 89.5 |
| Second autoclave | 90.0 |
| Third autoclave | 90.5 |

IBR-DORMIN® is heat stable. The small precipitates and the slight change in color observed after the autoclaving process, does not influence its activity.

### EXAMPLE 4

### Treatment with IBR DORMIN®

Plaque psoriasis of mild to moderate severity is routinely treated with topical steroids and coal tar, along with emollients. A safe and convenient new treatment modality would be of value to most patients suffering from psoriasis. A study was undertaken in order to assess the efficacy of 5% IBR-DORMIN® in the treatment of mild to moderate, persistent psoriasis. The results of the treatment of psoriasis in this study show that the left elbows of the patients (those treated with IBR-DORMIN®) exhibited a better overall improvement compared to their right elbows (treated with vehicle cream only). Additionally, no side effects were experienced on the IBR-DORMIN® treated elbow.

1. Methods

The application, twice daily, of 5% IBR-DORMIN® was compared with the application of its vehicle for up to 10 weeks in a double blind, controlled study of 15 patients, with no randomization. In the study, all of the patients applied 5% IBR-DORMIN® to one elbow and the vehicle cream to the other elbow.

Inclusion criteria for this study were as follows: all of the patients were between the ages of 16 and 70, and had mild to moderate stable psoriasis vulgaris. Exclusion criteria for this study included the presence of acute pruritus, acute urticaria, scabies, other systemic diseases that involve pruritus, steroidal treatment during the last month, pregnancy, treatment of systemic retinoids, and the use of any investigational drug within the last 30 days prior to study entry.

2. Results

The psoriatic plaques were judged by the clinical characteristics of thickness, dryness, desquamation, erythema, and pruritic lesions. These criteria were graded on a scale of 0 to 4, where 0=cure or absence and 4=severe. The clinical parameters were evaluated at baseline, 3, 6, and 10 weeks after start of the treatment regimen.

Two of the fifteen patients did not complete the study and follow-up of their conditions was lost. One patient did not come to the last follow-up visit (Visit #4), but was included in the study results. Total scoring parameter results for each patient at follow-up visits are summarized in Table 7.

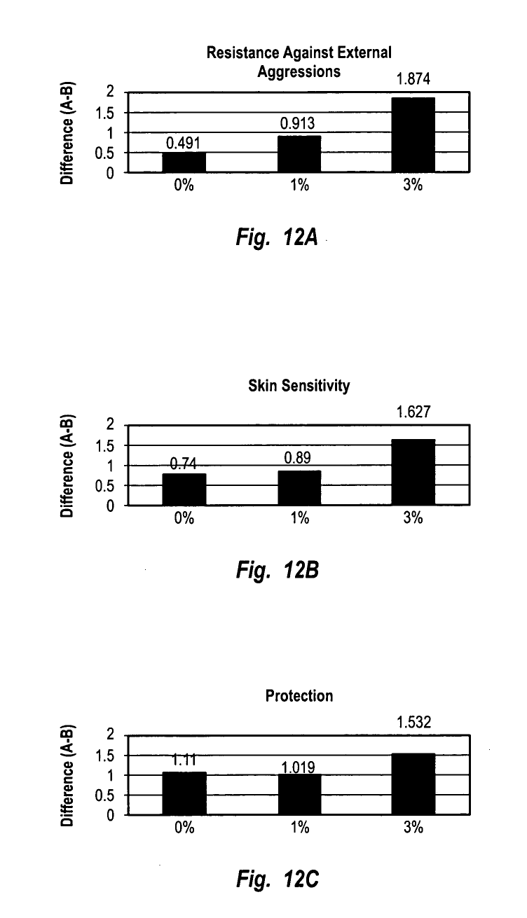

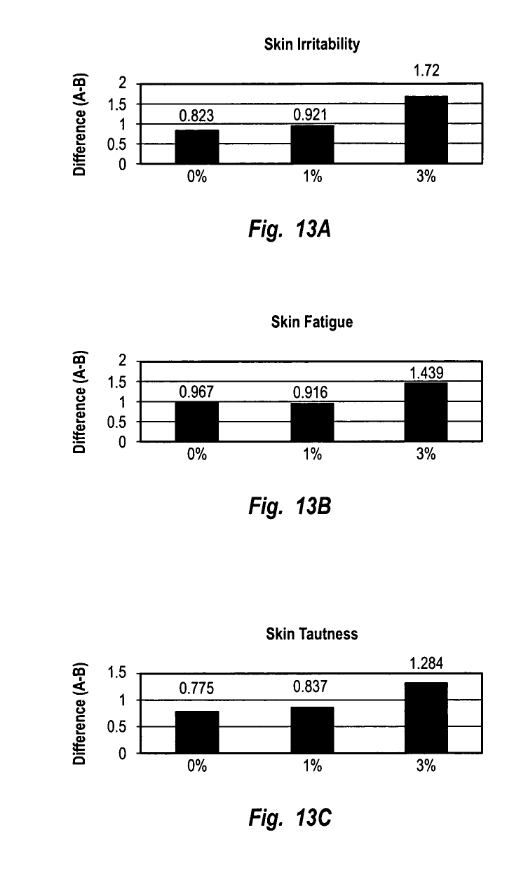

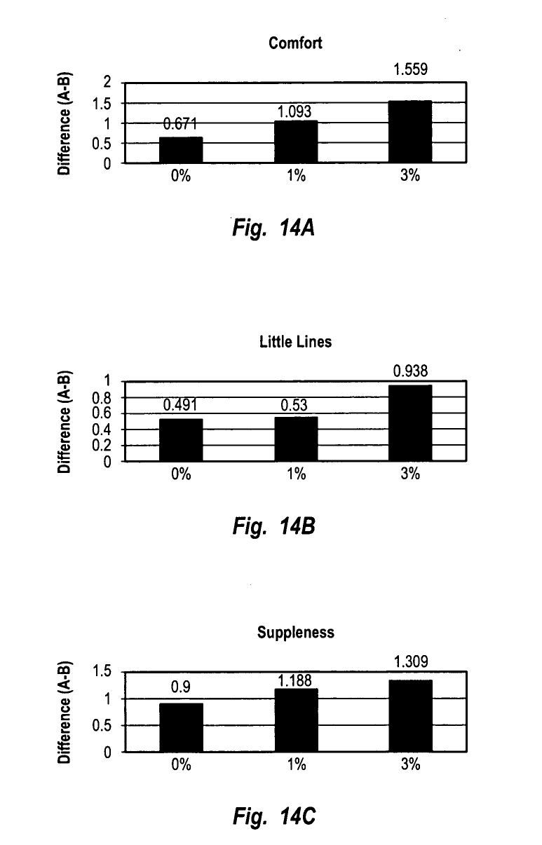

On the left elbow (IBR-DORMIN®): 3 patients had a complete cure; 2 patients had a cure rate of 75-99%; 4 patients had a cure rate of 50-74%; 3 patients had a cure rate of less than 50%, and in 1 patient, there was no change (FIGS. 13 and 14). On the right elbow (control):3 patients had a cure rate of 50-74%; 7 patients had a cure rate of less than 50%, and in 3 patients, there was no change (FIGS. 12 and 13).

When the study was completed, it was revealed that the left elbows were treated with 5% IBR-DORMIN® and the right elbows were treated with the vehicle cream only.

The results of the treatment of psoriasis in this study show that the left elbows of the patients (those treated with IBR-DORMIN®) exhibited a better overall improvement compared to their right elbows (treated with vehicle cream only). No side effects.

TABLE 7
Total Scoring Parameter Results for Each Patient at Follow-up Visits
Total Score of Clinical Parameters

| Patient Number | Visit Number | Right | Left |
|---|---|---|---|
| 1 | 1 | 8 | 10 |
| | 2 | 7 | 6 |
| | 3 | 4 | 5 |
| | 4 | 4 | 6 |
| 3 | 1 | 9 | 8 |
| | 2 | 7 | 2 |
| | 3 | 6 | 0 |
| | 4 | 6 | 0 |
| 4 | 1 | 9 | 6 |
| | 2 | 6 | 2 |
| | 3 | 5 | 0 |
| | 4 | 4 | 0 |
| 5 | 1 | 12 | 11 |
| | 2 | 12 | 4 |
| | 3 | 12 | 2 |
| | 4 | 11 | 2 |
| 6 | 1 | 10 | 11 |
| | 2 | 10 | 11 |
| | 3 | 10 | 11 |
| | 4 | 10 | 11 |
| 7 | 1 | 16 | 16 |
| | 2 | 14 | 6 |
| | 3 | 16 | 6 |
| | 4 | 16 | 6 |
| 8 | 1 | 8 | 8 |
| | 2 | 8 | 8˜ |
| | 3 | 4 | 2 |
| | 4 | Patient did | not show up |
| 10 | 1 | 15 | 15 |
| | 2 | 11 | 15 |
| | 3 | 11 | 15 |
| | 4 | 5 | 5 |
| 11 | 1 | 20 | 20 |
| | 2 | 20 | 20 |
| | 3 | 20 | 15 |
| | 4 | 20 | 12 |
| 12 | 1 | 15 | 15 |
| | 2 | 10 | 5 |
| | 3 | I1 | 0 |
| | 4 | 11 | 4 |
| 13 | 1 | 13 | 13 |
| | 2 | 13 | 13 |
| | 3 | 12 | 6 |
| | 4 | 12 | 4 |
| 4 | 1 | 15 | 20 |
| | 2 | 15 | 12 |
| | 3 | 5 | 10 |
| | 4 | 10 | 10 |

Legend:
Visit 1 = baseline.
Visit 2 = 3 weeks.
Visit 3 = 6 weeks.
Visit 4 = 10 weeks.

### EXAMPLE 5

### Treatment with IBR DORMIN®

1. Materials and Methods

150 women (mean age: 37.77±0.96) took part in the study. Three creams containing respectively 0% (placebo), 1% and 3% IBR-DORMIN® were produced for the study. IBR-DORMIN® was used to replace some of the water used in the formula for the excipient. The study lasted four weeks. The creams were applied daily by the women users themselves. 135 subjects completed the study; 48 in the placebo group (mean age: 36.71±1.53), 44 in the group testing the 1% cream (mean age: 37.93±1.7) and 43 in the 3% cream group (mean age: 38.77±1.8).

Rating scales were administered and handed in before the first application of the cream. Similar rating scales were administered at the end of the four weeks test. The parameters or items used in the test were the following: Resistance against external aggressions, Skin Sensitivity, Protection, Skin Irritability, Skin Fatigue, Skin Tautness, Comfort, Little Lines and Suppleness.

2. Data Analysis

Centimetric measures were drawn on unstructured scales 10 cm in length. The mean values before and after the cream application were obtained for each of the three groups. The before/after comparison was obtained statistically by a t-test, when applicable, using the SigmaStat 2.0 program. A Rank Sum Test (RST) was used each time the normality test failed using the same SigmaStat 2.0 program. The percentage of before/after change was calculated on the basis of means for each item.

3. Results: Resistance Against External Aggressions

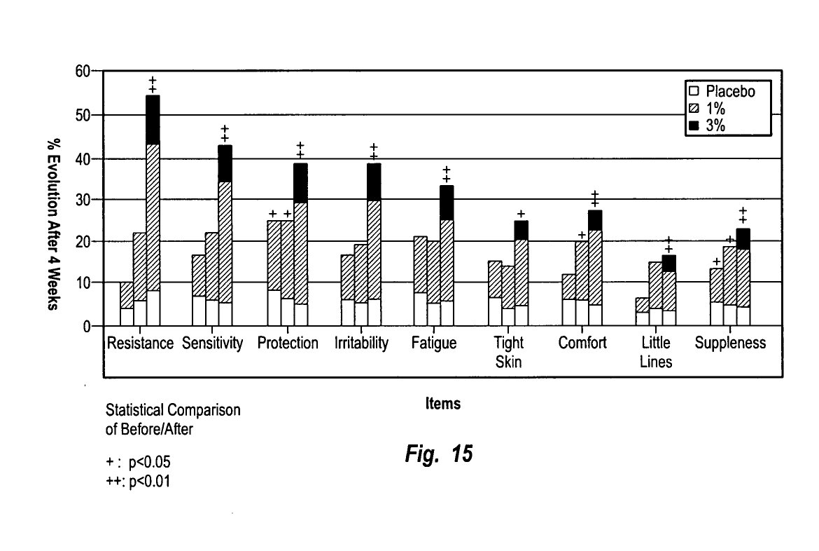

Results of the study for resistance against external aggressions are depicted in Table 8 and FIG. 15a. No statistical difference was found for the placebo and 1% IBR-DORMIN® cream. Significant statistical difference was found for the 3% IBR-DORMIN® cream.

TABLE 8
Resistance Against External Aggressions

| CRITERIA | PLACEBO | 1% CREAM | 3% CREAM |
|---|---|---|---|
| Number of women | 48 | 43 | 44 |
| Missing data | 0 | 2 | 1 |
| Mean before (B) | 4.690 ± 2.553 | 4.198 ± 2.627 | 3.414 ± 2.145 |
| Mean after (A) | 5.181 ± 2.445 | 5.112 ± 2.049 | 5.288 ± 2.165 |
| Difference (A − B) | 0.491 | 0.915 | 1.874 |
| Statistical test | RST | t-test | RST |
| Probability of | 0.395 | 0.083 | <0.001 |
| improvement (p) | | | |
| Significance | NS | NS | S (++) |

4. Results: Skin Sensitivity

Results for Skin sensitivity are depicted in Table 9 and FIG. 15b. No statistical difference was found for the placebo and 1% IBR-DORMIN® cream. Significant statistical difference was found for the 3% IBR-DORMIN® cream.

TABLE 9
Skin Sensitivity

| CRITERIA | PLACEBO | 1% CREAM | 3% CREAM |
|---|---|---|---|
| Number of women | 48 | 43 | 44 |
| Missing data | 1 | 1 | 0 |
| Mean before (B) | 4.294 ± 2.574 | 4.233 ± 2.396 | 3.745 ± 2.433 |
| Mean after (A) | 5.034 ± 2.477 | 5.124 ± 2.047 | 5.373 ± 2.180 |
| Difference (A − B) | 0.740 | 0.890 | 1.627 |
| Statistical test | t-test | t-test | t-test |
| Probability of | 0.159 | 0.07 | 0.003 |
| improvement (p) | | | |
| Significance | NS | NS | S (++) |

5. Results: Protection

Statistical results for protection assays are depicted in Table 10 and FIG. 15c. Statistical difference was found for the placebo and 1% IBR-DORMIN® cream. Significant statistical difference was found for the 3% IBR-DORMIN® cream.

TABLE 10
Protection Statistics

| CRITERIA | PLACEBO | 1% CREAM | 3% CREAM |
|---|---|---|---|
| Number of women | 48 | 43 | 44 |
| Missing data | 0 | 1 | 3 |
| Mean before (B) | 4.385 ± 2.203 | 4.112 ± 2.496 | 3.976 ± 2.425 |
| Mean after (A) | 5.496 ± 2.284 | 5.131 ± 2.087 | 5.507 ± 2.327 |
| Difference (A − B) | 1.11 | 1.019 | 1.532 |
| Statistical test | t-test | t-test | t-test |
| Probability of | 0.017 | 0.046 | 0.003 |
| improvement (p) | | | |
| Significance | S | S | S (++) |

6. Results: Skin Irritability

Statistical results for skin irritability are depicted in Table 11 and FIG. 16a. No statistical difference was found for the placebo and 1% IBR-DORMIN® cream. Significant statistical difference was found for the 3% IBR-DORMIN® cream.

TABLE 11
Skin Irritability Statistics

| CRITERIA | PLACEBO | 1% CREAM | 3% CREAM |
|---|---|---|---|
| Number of women | 48 | 43 | 44 |
| Missing data | 0 | 1 | 0 |
| Mean before (B) | 4794 ± 2.606 | 4.769 ± 2.336 | 4.405 ± 2.479 |
| Mean after (A) | 5.617 ± 2.372 | 5.690 ± 2.189 | 6.125 ± 2.128 |
| Difference (A − B) | 0.823 | 0.921 | 1.720 |
| Statistical test | t-test | t-test | t-test |
| Probability of | 0.109 | 0.066 | 0.001 |
| improvement (p) | | | |
| Significance | NS | NS | S (++) |

7. Results: Skin Fatigue

Statistical results for skin fatigue survey are depicted in Table 12 and FIG. 16b. No statistical difference was found for the placebo and 1% IBR-DORMIN® cream. Significant statistical difference was found for the 3% IBR-DORMIN® cream.

TABLE 12
Skin Fatiuge Statistics

| CRITERIA | PLACEBO | 1% CREAM | 3% CREAM |
|---|---|---|---|
| Number of women | 48 | 43 | 44 |
| Missing data | 0 | 1 | 0 |
| Mean before (B) | 4.635 ± 2.522 | 4.684 ± 2.374 | 4.345 ± 2.640 |
| Mean after (A) | 5.602 ± 2.265 | 5.600 ± 2.323 | 5.784 ± 2.017 |
| Difference (A − B) | 0.967 | 0.916 | 1.439 |
| Statistical test | t-test | t-test | t-test |
| Probability of | 0.051 | 0.074 | 0.005 |
| improvement (p) | | | |
| Significance | NS | NS | S (++) |

8. Results: Skin Tautness

Statistical results for skin tautness are depicted in Table 13 and FIG. 16c. No statistical difference was found for the placebo and 1% IBR-DORMIN® cream. Strong statistical difference was found for the 3% IBR-DORMIN® cream.

TABLE 13
Skin Tautness Statistics

| CRITERIA | PLACEBO | 1% CREAM | 3% CREAM |
|---|---|---|---|
| Number of women | 48 | 43 | 44 |
| Missing data | 0 | 0 | 0 |
| Mean before (B) | 5.158 ± 2.890 | 5.858 ± 2.497 | 5.173 ± 2.864 |
| Mean after (A) | 5.933 ± 2.713 | 6.695 ± 2.561 | 6.457 ± 2.636 |
| Difference (A − B) | 0.775 | 0.837 | 1.284 |
| Statistical test | t-test | RST | RST |
| Probability of | 0.179 | 0.092 | 0.032 |
| improvement (p) | | | |
| Significance | NS | NS | S |

9. Results: Skin Comfort

Statistical results for skin comfort survey are depicted in Table 14 and FIG. 17a. No statistical difference was found for the placebo. Significant statistical difference was found for the 1% IBR-DORMIN® cream. Significant statistical difference, at a higher probability, was found for the 3% IBR-DORMIN® cream.

TABLE 14
Skin Comfort Statistics

| CRITERIA | PLACEBO | 1% CREAM | 3% CREAM |
|---|---|---|---|
| Number of women | 48 | 43 | 44 |
| Missing data | 3 | 2 | 3 |
| Mean before (B) | 5.678 ± 2.059 | 5.410 ± 2.298 | 5.700 ± 1.934 |
| Mean after (A) | 6.349 ± 2.156 | 6.502 ± 1.940 | 7.259 ± 1.781 |
| Difference (A − B) | 0.671 | 1.093 | 1.559 |
| Statistical test | t-test | t-test | t-test |
| Probability of | 0.139 | 0.023 | <0.001 |
| improvement (p) | | | |
| Significance | NS | S | S |

10. Results: Little Lines

Statistical survey information related to little lines is depicted in Table 15 and FIG. 17b. No statistical difference was found for the placebo, 1% and 3% IBR-DORMIN® cream. However, note the relative young age of the women participated in the study (mean age: 37.77) and the values obtained that indicated few wrinkles. Another study with 3% IBR-DORMIN® cream used by women who put values indicating real lines showed an improvement of this item.

TABLE 15
Little Lines Statistics

| CRITERIA | PLACEBO | 1% CREAM | 3% CREAM |
|---|---|---|---|
| Number of women | 48 | 43 | 44 |
| Missing data | 0 | 0 | 0 |
| Mean before (B) | 6.583 ± 2.268 | 5.990 ± 2.582 | 5.939 ± 2.771 |
| Mean after (A) | 6.946 ± 2.062 | 6.867 ± 2.571 | 6.877 ± 2.361 |
| Difference (A − B) | 0.491 | 0.53 | 0.938 |
| Statistical test | RST | RST | RST |
| Probability of | 0.395 | 0.167 | 0.126 |
| improvement (p) | | | |
| Significance | NS | NS | NS |

11. Results: Suppleness

Statistical survey results related to skin suppleness are depicted in Table 16 and FIG. 17c. Statistical difference was found for the placebo. Significant statistical difference was found for the 1% and the 3% 18 IBR-DORMIN® creams.

TABLE 16
Suppleness Statistics

| CRITERIA | PLACEBO | 1% CREAM | 3% CREAM |
|---|---|---|---|
| Number of women | 48 | 43 | 44 |
| Missing data | 1 | 1 | 1 |
| Mean before (B) | 6.430 ± 1.978 | 6.174 ± 1.920 | 5.860 ± 2.045 |
| Mean after (A) | 7.330 ± 1.437 | 7.362 ± 1.520 | 7.170 ± 1.850 |
| Difference (A − B) | 0.9 | 1.188 | 1.309 |
| Statistical test | RST | t-test | t-test |
| Probability of | 0.022 | 0.002 | <0.001 |
| improvement (p) | | | |
| Significance | S | S (++) | S (++) |

12. Results: Percentage of Change

Statistical survey data related to percent change is depicted in Table 17 and FIG. 18.

TABLE 17
Evolution after 4 weeks [(after − before)/before × 100]

| ITEMS | PLACEBO | 1% CREAM | 3% CREAM |
|---|---|---|---|
| External | 17 | 22 | 55 |
| Aggressions | | | |
| Skin Sensitivity | 17 | 21 | 43 |
| Protection | 25 | 25 | 39 |
| Skin Irritability | 17 | 19 | 39 |
| Skin Fatigue | 21 | 20 | 33 |
| Skin Tautness | 15 | 14 | 25 |
| Comfort | 12 | 20 | 27 |
| Little Lines | 6 | 15 | 16 |
| Suppleness | 14 | 19 | 22 |

13. General Conclusions and Discussion

After four weeks of daily use, the findings suggest that the women users were able to recognize the cream containing IBR-DORMIN® as superior each time (FIG. 18). The effect was stronger for the group using the 3% preparation (Table 17). The most striking effects are related to skin resistance and protection, sensitivity and skin irritability. This could lend weight to the hypothesis that better maturation enables the skin to better fulfill its protective functions. Comfort, fatigue, tautness and suppleness also improved. There was no significant change as regards wrinkles in this experiment. However, as regards these parameters, note the relative youth of the women (mean age: 37.77) in the sample and the values indicating few wrinkles at the start of the study. According to the results obtained in this study IBR-DORMIN® at a concentration of 3% is best for cosmetic use.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims. Various publications are cited herein, the disclosures of which are incorporated by reference in their entireties.

1. A method for preventing damage to the skin, wherein said damage results from oxidative damage resulting from the generation of reactive oxygen species by arNOX, the method comprising:
administering to a patient an amount effective to prevent said damage, a composition comprising a processed Narcissus tazzeta product.

2. A method for treating damaged skin, wherein said damage results from oxidative damage resulting from the generation of reactive oxygen species by arNOX, the method comprising:
administering to a patient, in an amount effective to treat said damage, a composition comprising a processed Narcissus tazzeta product

3. The method of claim 1 or 2 wherein the composition comprises a Narcissus tazzeta extract, a preservative and water.

4. The method of claim 1 or 2 wherein the total daily amount of processed Narcissus tazzeta product administered is from about 1 to about 500 mg.

5. The method of claim 1 or 2 wherein the total daily amount of processed Narcissus tazzeta product administered is from about 1 to about 100 mg.

6. The method of claim 1 or 2 wherein the composition further comprises a ubiquinone.

7. The method of claim 6, wherein the ubiquinone is coenzyme Q₁₀.

8. The method of claim 7 wherein coenzyme Q₁₀ is administered with a ubiquinone selected from a group consisting of coenzyme Q₅, coenzyme Q₇, coenzyme Q₈, and coenzyme Q₉.

9. The method of claim 1 or 2, wherein the composition further an ingredient selected from a list comprising Shisandra Chinensis, Lonicera Japonica, Fagopyrum Cymosum, Methlyparaben, L-Carnosine, Propylparaben, Ethylparaben, L-Ergothioneine, Betulinic acid, Solanum Lycopersicum, Univestin, and Soliprin.

10. The method of claim 1 or 2 wherein the damage is a result of a primary disorder selected from a list comprising: old age, rheumatoid arthritis, cancer arthritis associated with age, and fatigue associated with age.

11. The method of claim 1 or 2 wherein the damage is a result of aged cells.

12. The method of claim 1 or 2, wherein the damage is a result of a dermatological disorder selected from a list comprising: Acne Vulgaris, Adiposis Dolorosa, Albinism, Alopecia, alpha 1-Antitrypsin Deficiency, Atopic dermatitis, Baldness, Behcet's Syndrome, Birthmarks, Birt-Hogg-Dube Syndrome, Blister, Cafe-au-Lait Spots, Cellulitis, Cholesteatoma, Connective Tissue Diseases, Contractural Arachnodactyly, Cutis Laxa, Decubitus Ulcer, Dercum Disease, Dermatitis, Dermatitis Exfoliative, Dermatitis Herpetiformis, Ectodermal Dysplasia, Eczema, Ehlers-Danlos Syndrome, Epidermolysis Bullosa, Erysipelas, Erythema Multiforme, Exanthema Subitum, Furunculosis, Granuloma Annulare, Gustatory Sweating, Hailey-Hailey Disease, Hair Diseases, Hair Loss, Head Lice, Hidradenitis Suppurativa, Hirsutism, Hives, Hypohidrosis, Ichthyosis, Immersion Foot, Incontinentia Pigmenti, Keloid, Keratosis Actinic, Keratosis Follicularis, Keratosis Seborrheic, Leg Ulcer, Lentigo, Lichen Planus, Lichen Sclerosus et Atrophicus, Lipodystrophy, Lupus, Lupus Erythematosus Cutaneous, Lupus Erythematosus Systemic, Marfan Syndrome, Mastocytosis. Melanoma, Melanosis, Mixed Connective Tissue Disease, Nail Patella Syndrome, Nail Diseases, Nails Ingrown, Panniculitis, Parapsoriasis, Paronychia, Pemphigoid Bullous, Pemphigus, Pemphigus Benign Familial, Photosensitivity Disorders, Pigmentation Disorders, Pityriasis, Poison Ivy, Port-Wine Stain, Pruritus, Pseudoxanthoma Elasticum, Psoriasis, Pyoderma Gangrenosum, Rosacea, Scabies, Scleroderma, Scleroderma Systemic, Seborrheic Dermatitis, Shopping, Skin Cancer, Skin and Connective Tissue Diseases, Skin Diseases, Infectious Skin Diseases, Skin Ulcer, Stevens-Johnson Syndrome, Stickler Syndrome, Sweat Gland Diseases, Sweet's Syndrome, Swimmer's Itch, Tinea Versicolor, Urticaria, Vitiligo, Warts, Xanthomatosis, and Xeroderma Pigmentosum.

13. The method of claim 1 or 2 wherein the damage is selected from a list comprising wrinkles, fine lines, large pore size, acne, excessive sebum production, collagen damage, elastin damage and damaged fibroblasts.

14. A method of preventing a complication of a primary disorder in patients wherein said complication results from oxidative damage resulting from the generation of reactive oxygen species by arNOX, the method comprising:
administering to a patient having said primary disorder, in an amount effective to prevent said complication, a composition comprising a processed Narcissus tazzeta product.

15. A method for preventing secondary disorders in patients having a primary disorder that causes oxidative damage resulting from the generation of reactive oxygen species by arNOX, the method comprising:
administering to a patient having said primary disorder, in an amount effective to prevent said complication, a composition comprising a processed Narcissus tazzeta product.

16. The method of claim 14 or 15 wherein the composition comprises a Narcissus tazzeta extract, a preservative and water.

17. The method of claim 14 or 15 wherein the total daily amount of processed Narcissus tazzeta product administered is from about 1 to about 500 mg.

18. The method of claim 14 or 15 wherein the total daily amount of processed Narcissus tazzeta product administered is from about 1 to about 100 mg.

19. The method of claim 14 or 15 wherein the composition further comprises a ubiquinone.

20. The method of claim 19, wherein the ubiquinone is coenzyme Q₁₀.

21. The method of claim 20 wherein coenzyme Q₁₀ is administered with a ubiquinone selected from a group comprising coenzyme Q₅, coenzyme Q₇, coenzyme Q₈, and coenzyme Q₉.

22. The method of claim 14 or 15, wherein the composition further comprises an ingredient selected from a list comprising Shisandra Chinensis, Lonicera Japonica, Fagopyrum Cymosum, methlyparaben, L-Camosine, Propylparaben, Ethylparaben, L-Ergothioneine, Betulinic acid, Solanum Lycopersicum, Univestin, and Soliprin.

23. The method of claim 14 or 15 wherein the primary disorder is old age, rheumatoid arthritis, arthritis associated with age, or fatigue associated with age.

24. The method of claim 14 or 15 wherein the primary disorder is a result of aged cells.

25. The method of claim 14 or 15 wherein the primary disorder is cancer.

26. The method of claim 14 or 15 wherein the primary disorder is selected from a group comprising myocardial infarction, alcoholism, favism, malaria, sickle cell anemia, Fanconi's anemia, protoporphyrin photo-oxidation, nutritional deficiencies, Kwashiorkor, thalassemia, dietary iron overload, idiopathic hemochromatosis, metal ion-mediated nephrotoxicity, aminoglycoside nephrotoxicity, autoimmune nephrotic syndromes, oral iron poisoning, endotoxin liver injury, free fatty acid-induced pancreatitis, nonsteroidal anti-inflammatory drug induced gastrointestinal tract lesions, glomerulonephritis, autoimmune diseases, vasculitis, hepatitis B virus, Parkinson's disease, neurotoxins, allergic encephalomyelitis, potentiation of traumatic injury, hypertensive cerebrovascular injury, vitamin B deficiency, adriamycin cardiotoxicity, Keshan disease, selenium deficiency, alcohol cardiomyopathy, photic retinopathy, ocular hemorrhage, cataractogenesis, degenerative retinal damage, amyotrophic lateral sclerosis, age-related macular degeneration, diabetes, atherogenesis, atherosclerosis, Acne Vulgaris, Adiposis Dolorosa, Albinism, Alopecia, alpha 1-Antitrypsin Deficiency, Atopic dermatitis, Baldness, Behcet's Syndrome, Birthmarks, Birt-Hogg-Dube Syndrome, Blister, Cafe-au-Lait Spots, Cellulitis, Cholesteatoma, Connective Tissue Diseases, Contractural Arachnodactyly, Cutis Laxa, Decubitus Ulcer, Dercum Disease, Dermatitis, Dermatitis Exfoliative, Dermatitis Herpetiformis, Ectodermal Dysplasia, Eczema, Ehlers-Danlos Syndrome, Epidermolysis Bullosa, Erysipelas, Erythema Multiforme, Exanthema Subitum, Furunculosis, Granuloma Annulare, Gustatory Sweating, Hailey-Hailey Disease, Hair Diseases, Hair Loss, Head Lice, Hidradenitis Suppurativa, Hirsutism, Hives, Hypohidrosis, Ichthyosis, Immersion Foot, Incontinentia Pigmenti, Keloid, Keratosis Actinic, Keratosis Follicularis, Keratosis Seborrheic, Leg Ulcer, Lentigo, Lichen Planus, Lichen Sclerosus et Atrophicus, Lipodystrophy, Lupus, Lupus Erythematosus Cutaneous, Lupus Erythematosus Systemic, Marfan Syndrome, Mastocytosis, Melanoma, Melanosis, Mixed Connective Tissue Disease, Nail Patella Syndrome, Nail Diseases, Nails Ingrown, Panniculitis, Parapsoriasis, Paronychia, Pemphigoid Bullous, Pemphigus, Pemphigus Benign Familial, Photosensitivity Disorders, Pigmentation Disorders, Pityriasis, Poison Ivy, Port-Wine Stain, Pruritus, Pseudoxanthoma Elasticum, Psoriasis, Pyoderma Gangrenosum, Rosacea, Scabies, Scleroderma, Scleroderma Systemic, Seborrheic Dermatitis, Shopping, Skin Cancer, Skin and Connective Tissue Diseases, Skin Diseases, Infectious Skin Diseases, Skin Ulcer, Stevens-Johnson Syndrome, Stickler Syndrome, Sweat Gland Diseases, Sweet's Syndrome, Swimmer's Itch, Tinea Versicolor, Urticaria, Vitiligo, Warts, Xanthomatosis, and Xeroderma Pigmentosum.

27. A method of inhibiting the generation of reactive oxygen species by arNOX, comprising the step of: administering to a patient an effective amount of a composition comprising an ingredient selected from a list comprising: processed Narcissus tazzeta product, a ubiquinone, coenzyeme Q₁₀, coenzyme Q₅, coenzyme Q₇, coenzyme Q₈, coenzyme Q₉, Shisandra Chinensis, Lonicera Japonica, Fagopyrum Cymosum, methlyparaben, L-Camosine, Propylparaben, Ethylparaben, L-Ergothioneine, Betulinic acid, Solanum Lycopersicum, Univestin, and Soliprin.

28. A method for sequestering arNOX in a patient, the method comprising administering to a patient an effective amount of a composition comprising an ingredient selected from a list comprising a processed Narcissus tazzeta product, a ubiquinone, coenzyeme Q₁₀, coenzyme Q₅, coenzyme Q₇, coenzyme Q₈, coenzyme Q₉, Shisandra Chinensis, Lonicera Japonica, Fagopyrum Cymosum, methlyparaben, L-Camosine, Propylparaben, Ethylparaben, L-Ergothioneine, Betulinic acid, Solanum Lycopersicum, Univestin, and Soliprin.

29. A method of preventing a complication of a primary disorder in patients wherein said complication results from oxidative damage resulting from the generation of reactive oxygen species by arNOX, the method comprising:
administering to a patient having said primary disorder, in an amount effective to prevent said complication, a composition comprising an ingredient selected from a list comprising: Shisandra Chinensis, Lonicera Japonica, Fagopyrum Cymosum, methlyparaben, L-Carnosine, Propylparaben, Ethylparaben, L-Ergothioneine, Betulinic acid, Solanum Lycopersicum, Univestin, Soliprin, a processed Narcissus tazzeta product, a ubiquinone, coenzyeme Q₁₀, coenzyme Q₅, coenzyme Q₇, coenzyme Q₉, and coenzyme Q₉.

30. A method for preventing secondary disorders in patients having a primary disorder that causes oxidative damage resulting from the generation of reactive oxygen species by arNOX, the method comprising:
administering to a patient having said primary disorder, in an amount effective to prevent said complication, a composition comprising Shisandra Chinensis, Lonicera Japonica, Fagopyrum Cymosum, methlyparaben, L-Carnosine, Propylparaben, Ethylparaben, L-Ergothioneine, Betulinic acid, Solanum Lycopersicum, Univestin, Soliprin, a processed Narcissus tazzeta product, a ubiquinone, coenzyeme Q₁₀, coenzyme Q₅, coenzyme Q₇, coenzyme Q₈, or coenzyme Q₉.

31. A method of screening for agents that sequester arNOX, comprising:
(a) incubating arNOX with a test agent for a time sufficient to allow the test agent to bind arNOX; and
(b) detecting the presence of a complex comprising arNOX and the test agent.

32. A method of screening for agents that sequester arNOX comprising:
(a) incubating arNOX with a test agent, cytochrome c, and a substrate that generates reactive oxygen species, for a time sufficient for cytochrome c reduction; and
(b) detecting the presence of reduced cytochrome c, in the presence or absence of the test agent, whereas the absence of reduced cytochrome c in the mixture comprising the test agent indicates that the test agent sequesters arNOX.

33. A method of screening for agents that sequester arNOX comprising:
(a) incubating arNOX with a test agent and a substrate, wherein said substrate is reduced by arNOX, for a time sufficient for arNOX to reduce said substrate; and
(b) detecting the presence of reduced substrate in the presence or absence of the test agent, whereas the absence of reduced substrate in the mixture comprising the test agent indicates that the test agent sequesters arNOX.

34. A method of screening for agents that sequester arNOX comprising
(a) incubating arNOX with a test agent and a substrate, wherein said substrate undergoes disulfide-thiol interchange activity in the presence of arNOX, for a time sufficient for arNOX to reduce said substrate; and
(b) detecting the presence of disulfide-thiol interchange in the substrate in the presence or absence of the test agent, whereas the absence of disulfide-thiol interchange in the substrate in the mixture comprising the test agent indicates that the test agent sequesters arNOX.

35. A method of preventing a complication of a primary disorder in patients wherein said complication results from oxidative damage resulting from the generation of reactive oxygen species by arNOX, which comprises:
administering to a patient having said primary disorder, in an amount effective to prevent said complication, an agent that sequesters arNOX, identified by the methods of claim 31, 32, 33, or 34.

36. A method for preventing secondary disorders in patients having a primary disorder that causes oxidative damage resulting from the generation of reactive oxygen species by arNOX, which comprises:
administering to a patient having a primary disorder, in an amount effective to prevent said secondary disorder, an agent that sequesters arNOX, identified by the methods of claim 31, 32, 33, and 34.

37. A composition for preventing damage to the skin, wherein said damage results from oxidative damage resulting from the generation of reactive oxygen species by arNOX, the composition comprising:
a processed Narcissus tazzeta product.

38. A composition for treating damaged skin, wherein said damage results from oxidative damage resulting from the generation of reactive oxygen species by arNOX, the composition comprising:
a processed Narcissus tazzeta product

39. A composition for preventing a complication of a primary disorder in patients wherein said complication results from oxidative damage resulting from the generation of reactive oxygen species by arNOX, the composition comprising:
a processed Narcissus tazzeta product.

40. A composition for preventing secondary disorders in patients having a primary disorder that causes oxidative damage resulting from the generation of reactive oxygen species by arNOX, the composition comprising:
a processed Narcissus tazzeta product.

41. A composition for inhibiting the generation of reactive oxygen species by arNOX, comprising an ingredient selected from a list consisting of: a processed Narcissus tazzeta product, a ubiquinone, coenzyeme Q₁₀, coenzyme Q₅, coenzyme Q₇, coenzyme Q₈, coenzyme Q₉, Shisandra Chinensis, Lonicera Japonica, Fagopyrum Cymosum, methlyparaben, L-Carnosine, Propylparaben, Ethylparaben, L-Ergothioneine, Betulinic acid, Solanum Lycopersicum, Univestin, and Soliprin.

42. A composition for sequestering arNOX in a patient comprising an ingredient selected from a list consisting of: a processed Narcissus tazzeta product, a ubiquinone, coenzyeme Q₁₀, coenzyme Q₅, coenzyme Q₇, coenzyme Q₈, coenzyme Q₉, Shisandra Chinensis, Lonicera Japonica, Fagopyrum Cymosum, methlyparaben, L-Carnosine, Propylparaben, Ethylparaben, L-Ergothioneine, Betulinic acid, Solanum Lycopersicum, Univestin, and Soliprin.

43. A composition for preventing a complication of a primary disorder in patients wherein said complication results from oxidative damage resulting from the generation of reactive oxygen species by arNOX, the composition comprising:
an ingredient selected from a list consisting of: Shisandra Chinensis, Lonicera Japonica, Fagopyrum Cymosum, methlyparaben, L-Camosine, Propylparaben, Ethylparaben, L-Ergothioneine, Betulinic acid, Solanum Lycopersicum, Univestin, Soliprin, a processed Narcissus tazzeta product, a ubiquinone, coenzyeme Q₁₀, coenzyme Q₅, coenzyme Q₇, coenzyme Q₈, and coenzyme Q₉.

44. A composition for preventing secondary disorders in patients having a primary disorder that causes oxidative damage resulting from the generation of reactive oxygen species by arNOX, the composition comprising:
an active agent selected from a list consisting of: Shisandra Chinensis, Lonicera Japonica, Fagopyrum Cymosum, methlyparaben, L-Carnosine, Propylparaben, Ethylparaben, L-Ergothioneine, Betulinic acid, Solanum Lycopersicum, Univestin, Soliprin, a processed Narcissus tazzeta product, a ubiquinone, coenzyeme Q₁₀ coenzyme Q₅, coenzyme Q₇, coenzyme Q₉, and coenzyme Q₉.

45. A composition for treating damaged skin, wherein said damage results from oxidative damage resulting from the generation of reactive oxygen species by arNOX consisting essentially of an ingredient selected from a list consisting of: Shisandra Chinensis, Lonicera Japonica, Fagopyrum Cymosum, methlyparaben, L-Carnosine, Propylparaben, Ethylparaben, L-Ergothioneine, Betulinic acid, Solanum Lycopersicum, Univestin, Soliprin, a processed Narcissus tazzeta product, a ubiquinone, coenzyeme Q₁₀, coenzyme Q₅, coenzyme Q₇, coenzyme Q₈, and coenzyme Q₉.
